(19) United States
(12) Patent Application Publication
(10) Pub. No.: US 2013/0303479 A1
(43) Pub. Date: Nov. 14, 2013

(54) Antiretroviral Cyclonucleoside Compositions and Methods and Articles of Title of Invention Manufacture Therewith

(71) Applicants: Todd William Hawley, Reston, VA (US); Yuntao Wu, Manassas, VA (US); Jia Guo, Manassas, VA (US)

(72) Inventors: Todd William Hawley, Reston, VA (US); Yuntao Wu, Manassas, VA (US); Jia Guo, Manassas, VA (US)

(21) Appl. No.: 13/980,566

(22) PCT Filed: Jan. 19, 2012
(86) PCT No.: PCT/US2012/021817

§ 371 Date: Jul. 18, 2013

Related U.S. Application Data
(60) Provisional application No. 61/434,489, filed on Jan. 20, 2011.

(51) Int. Cl. A61K 31/519; A61K 45/06; A61K 31/7072; A61K 31/506

(52) U.S. Cl. CPC A61K 31/519; A61K 31/506; A61K 45/06; A61K 31/7072
USPC 514/50; 514/267

(57) ABSTRACT

ASPECTS OF EMBODIMENTS RELATE TO methods of treating human immunodeficiency virus (HIV) infection. Further aspects of embodiments also relate to constellations of compositions for treating HIV infection. Still additional aspects of embodiments relate to a many methods of making compositions useful in the treatment of HIV infection.

This application claims the benefit of U.S. Provisional Application No. 61/434,489, filed 20 Jan. 2011, which is hereby incorporated by reference in its entirety.

### BRIEF DESCRIPTION OF THE SEVERAL VIEWS OF THE DRAWINGS

FIG. 1 is a scatterplot depicting the results of a luciferase reporter assay as per an aspect of an embodiment of the present invention.

FIG. 2 is a bar chart depicting the results of a drug dilution assay with O2,2′-cyclocytidine monoacetate (Cyclo-C) (NSC129220) treated GFP Rev dependent reporter cells infected with similar amounts of HIV-1 wildtype (wt) as per an aspect of an embodiment of the present invention.

FIG. 3 is a dose-response curve depicting the effects various concentrations of O2,2′-cyclocytidine monoacetate (Cyclo-C) (NSC129220) at on HIV Rev dependent luciferase reporter cells infected with similar amounts of HIV as per an aspect of an embodiment of the present invention.

FIG. 4 is a dose-response curve comparing the effects of various concentrations 5-fluorocyclocytidine (AAFC) (NSC 166641) (“Flori”), O2,2′-cyclocytidine monoacetate (Cyclo-C) (NSC129220) and O2,2′-cyclocytidine hydrochloride (Cyclo-C)(NSC 145668) at on HIV Rev dependent luciferase reporter cells infected with similar amounts of HIV as per an aspect of an embodiment of the present invention.

FIG. 5 is a dose-response curve comparing the effects of Flori and O2,2′-cyclocytidine monoacetate (Cyclo-C) at various concentrations on HIV Rev dependent luciferase reporter cells infected with similar amounts of HIV as per an aspect of an embodiment of the present invention.

FIG. 6 is a graph showing the results of various concentrations of Flori performed on CEM-SS cells with an ATP based cell titer toxicity normalization assay as per an aspect of an embodiment of the present invention.

FIG. 7 is a graph showing the dose response effects of 5-fluoro-2,2′cyclocytidine (AAFC), on peripheral blood mononuclear cells infected with the HIV-1 wildtype (wt) virus and analyzed via p24 ELISA as per an aspect of an embodiment of the present invention.

FIG. 8 is a bar chart depicting the effects of Flori on total HIV-1 (wt) DNA copy number as determined by real time polymerase chain reaction (RT-PCR) as per an aspect of an embodiment of the present invention.

FIG. 9 is a bar chart depicting the effects of various concentrations of Flori on reverse transcriptase (RT) activity as per an aspect of an embodiment of the present invention.

FIG. 10 is a graph depicting the cellular growth inhibition as measured by the presence of ATP of various concentrations of Flori on human peripheral blood mononuclear cells from donor (x76) as per an aspect of an embodiment of the present invention.

FIG. 11 is a graph depicting the effects of various concentrations of Flori on human peripheral blood mononuclear cells over stimulated with IL-2 in combination with PHA and cultured in enriched media (20% FBS) and exposed to similar amounts of HIV-1 wildtype virus as per an aspect of an embodiment of the present invention.

FIG. 12 is a graph depicting GFP Rev dependent reporter cells infected with similar amounts of HIV-1 (wt) and then treated at various times after infection with Flori as per an aspect of an embodiment of the present invention.

FIG. 13 is a graph depicting the results of a Flori drug dilution assay with G11 GFP HIV Rev dependent reporter cells exposed to similar amounts of HIV-1 wild type as per an aspect of an embodiment of the present invention.

FIG. 14 is a graph of luciferase activity in the presence of deoxythymidine (dT) and deoxycytidine (dC) as per an aspect of an embodiment of the present invention.

FIG. 15 is a dose response graph of HIV Rev dependent luciferase reporter cells exposed to O2,2′-cyclocytidine monoacetate (Cyclo-C) (NSC129220), 200 uM deoxycytidine and exposed to HIV-1 pseudotyped virus as per an aspect of an embodiment of the present invention.

FIG. 16 is a dose response graph of HIV Rev dependent luciferase reporter cells exposed to Flori 200 uM deoxycytidine and exposed to HIV-1 pseudotyped virus as per an aspect of an embodiment of the present invention.

FIG. 17 is a graph of HIV Rev dependent luciferase reporter cells exposed to 10 μM Flori and dosages of deoxythymidine, deoxycytidine or a combination of both as per an aspect of an embodiment of the present invention.

FIG. 18 is a graph of HIV Rev dependent luciferase reporter cells exposed to 1 μM Flori and dosages of deoxythymidine (dT), deoxycytidine (dC) or a combination of both as per an aspect of an embodiment of the present invention.

FIG. 19 is a stick diagram depicting common structural features of cyclocytidines as per an aspect of an embodiment of the present invention.

FIGS. 20-25 depict structural features of additional cyclocytidines as per an aspect of an embodiment of the present invention.

FIG. 26 (panels a-d) depicts four graphs depicting the dose-dependent effect of Flori as per an aspect of an embodiment of the present invention.

FIG. 27 depicts the results showing the effectiveness of the cyclocytidines compound in a fluorescence activated cell sorting (FACS) green fluorescent protein (GFP) assay showing cell viability >60% for HIV Rev dependent GFP reporter cells treated with O2,2′-cyclocytidine monoacetate (Cyclo-C) (NSC129220) as per an aspect of an embodiment of the present invention.

FIG. 28 contains four line graphs depicting the dose-dependent effect of Flori on p24 levels in HIV-infected PBMC at 3, 6, 9 and 12 days as per an aspect of an embodiment of the present invention.

FIG. 29 depicts the dose-dependent effect of Flori on p24 levels in PMBC cells infected with similar amounts of HIV-wt and then stimulated every other day with IL-2 combined with PHA and grown in enriched (+20% FBS) medium as per an aspect of an embodiment of the present invention.

FIG. 30 depicts a scatter plot showing Flori and the entire set of 1,364 compounds tested in an anti-HIV screen using a Rev dependent luciferase reporter cell line, as indicated by the quenching of luminescence in cells as per an aspect of an embodiment of the present invention.

FIG. 31 depicts a head-to-head comparison in Rev dependent luciferase reporter cells infected with HIV pseudotyped virus and treated with the indicated concentrations of AZT, an approved anti-HIV drug or with Flori per an aspect of an embodiment of the present invention.

FIG. 32 (panels a-b) depicts a head-to-head comparison of several approved anti-HIV drugs and metabolic inhibitors with Flori in a rev dependent luciferase reporter cell line as per an aspect of an embodiment of the present invention.

FIG. 33 (panels a-b) depicts graphs comparing the relative potency and cooperation of Flori and the FDA approved antiretroviral drug Lamivudine (3TC) when separately or concomitantly contacted with cells as per an aspect of an embodiment of the present invention.

FIG. 34 is a bar graph depicting p24 ELISA analysis of the effects of the indicated amounts of Flori on blood active resting CD4 T-cells infected with HIV-wt, stimulated on day 5 with CD3/CD28 beads to activate latent virus as per an aspect of an embodiment of the present invention.

FIG. 35 depicts a Flori (AAFC)—dCK inhibition curve as per an aspect of an embodiment of the present invention.

### DETAILED DESCRIPTION

Embodiments relate to inhibitors of deoxycytidine kinase and processes therewith. Some embodiments relate to articles of manufacture useful in treating disease, a method of making therapeutic compositions, combinations of therapeutic compositions, methods of administering therapeutic compositions, and dosages of therapeutic compositions.

According to embodiments, a process to treat human immunodeficiency virus (HIV) infection may include administering to the patient a therapeutically effective amount of an inhibitor of deoxycytidine kinase. In embodiments, deoxycytidine kinase inhibitors may include cyclocytidine, pharmaceutically acceptable salts of O2,2′-cyclocytidine, O2,2′-cyclocytidine monoacetate, O2,2′-cyclocytidine hydrochloride, pharmaceutically acceptable salts of 5-fluoro-2, 2′cyclocytidine and 5-fluoro-2,2′cyclocytidine.

Embodiments relate to methods of using cyclocytidine compounds to inhibit viral, fungal, and bacterial replication. Embodiments also relate to methods of inhibiting HIV replication.

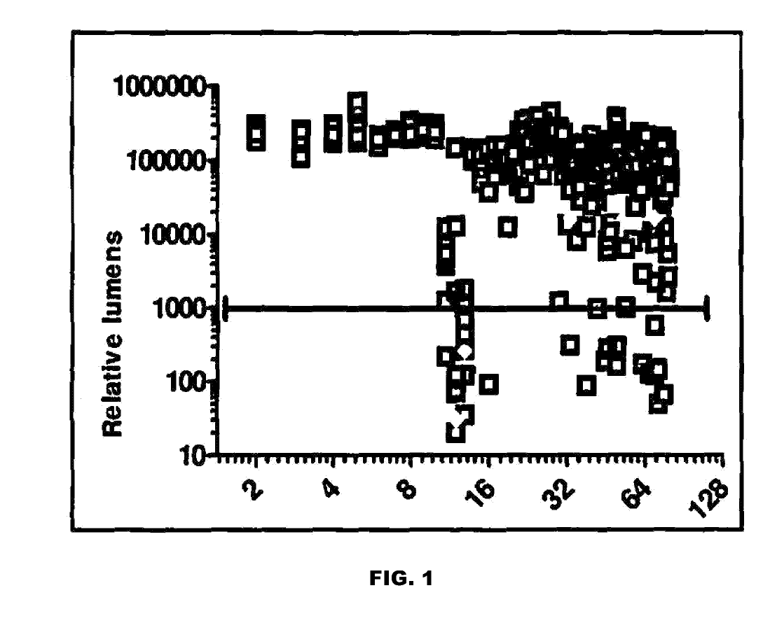

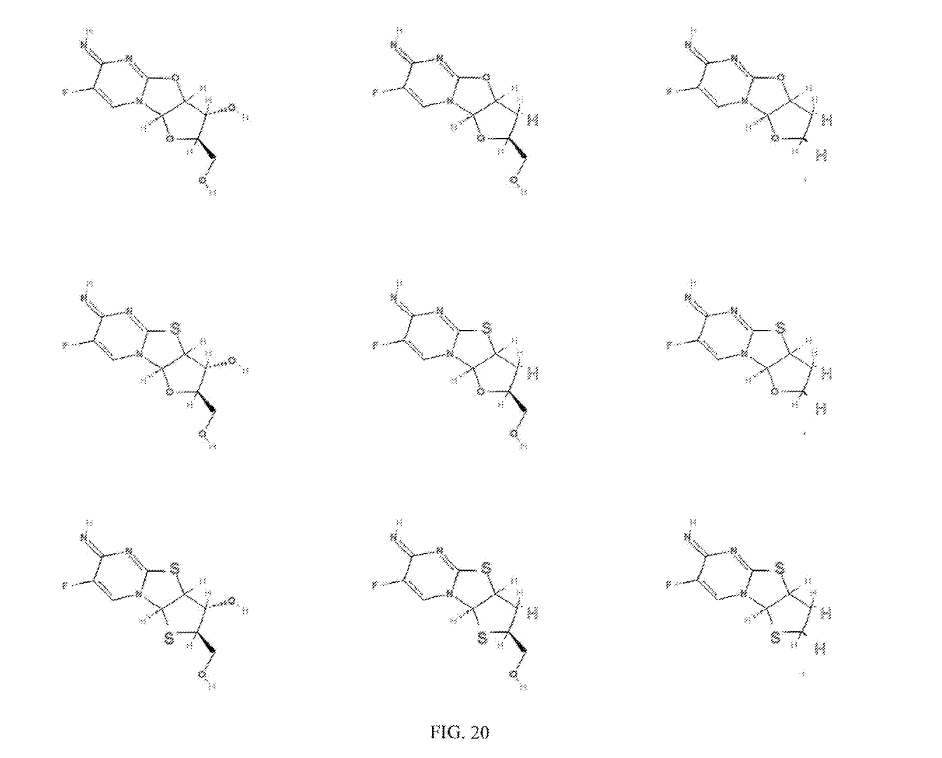

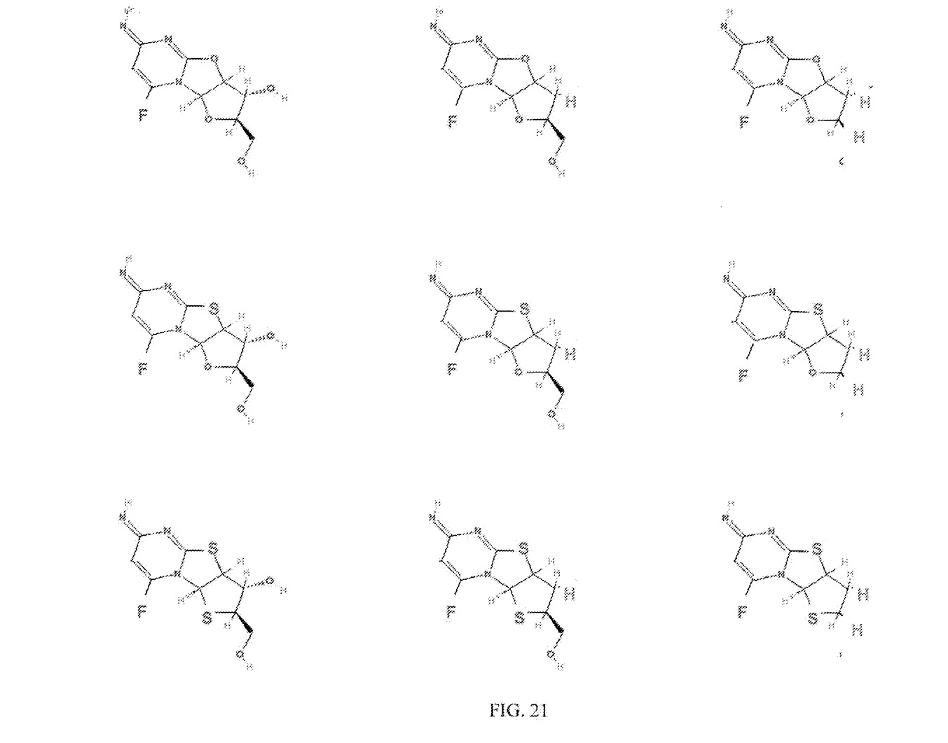

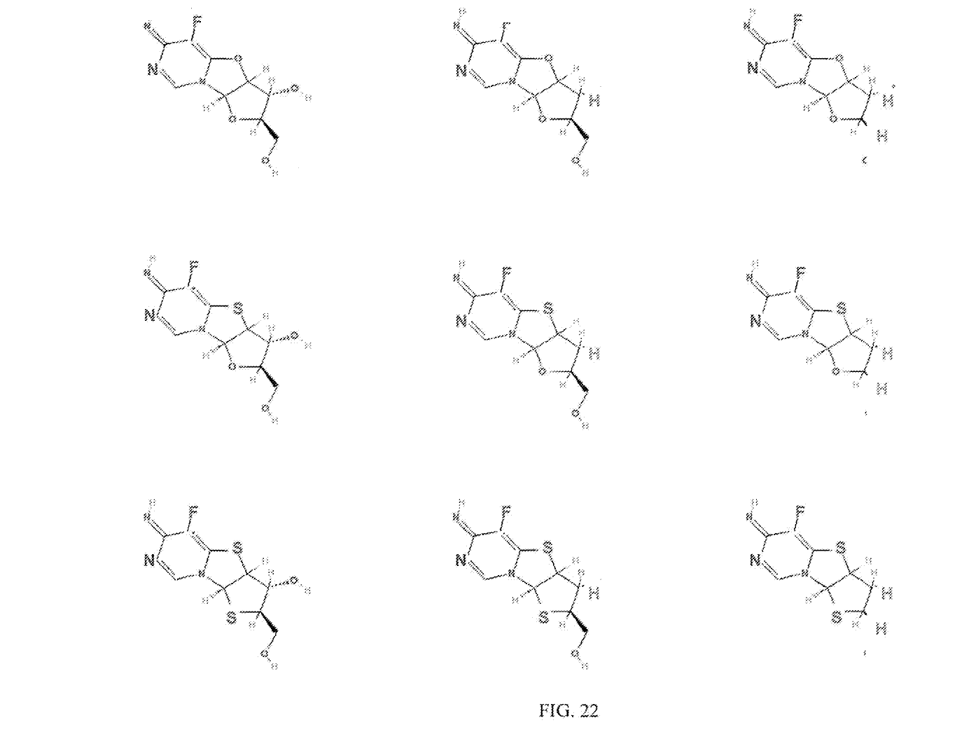

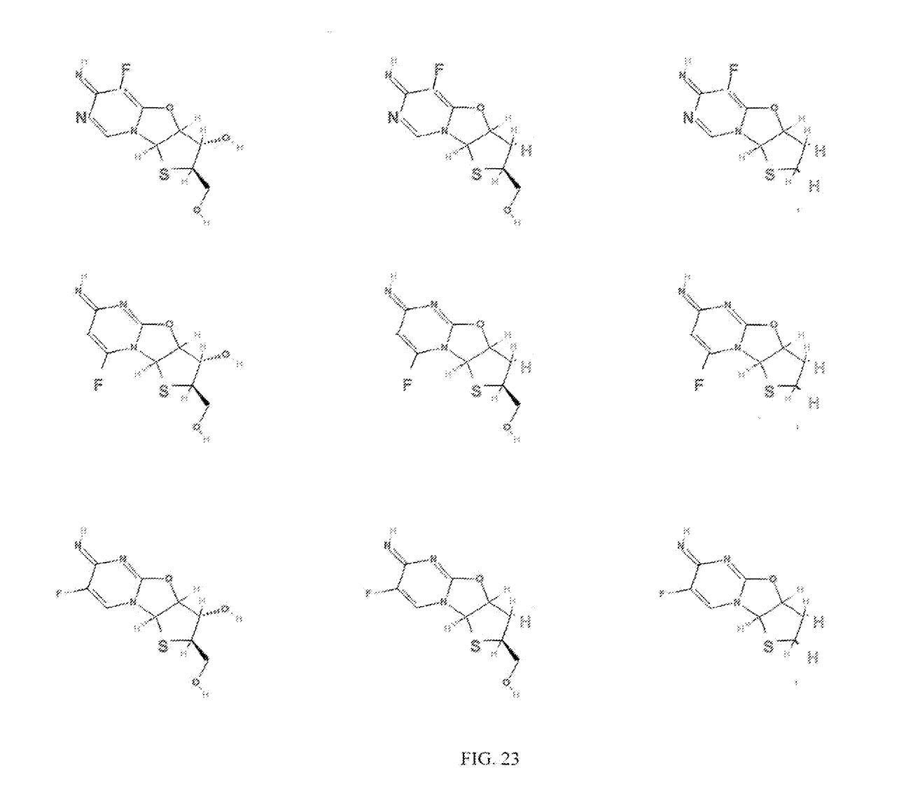

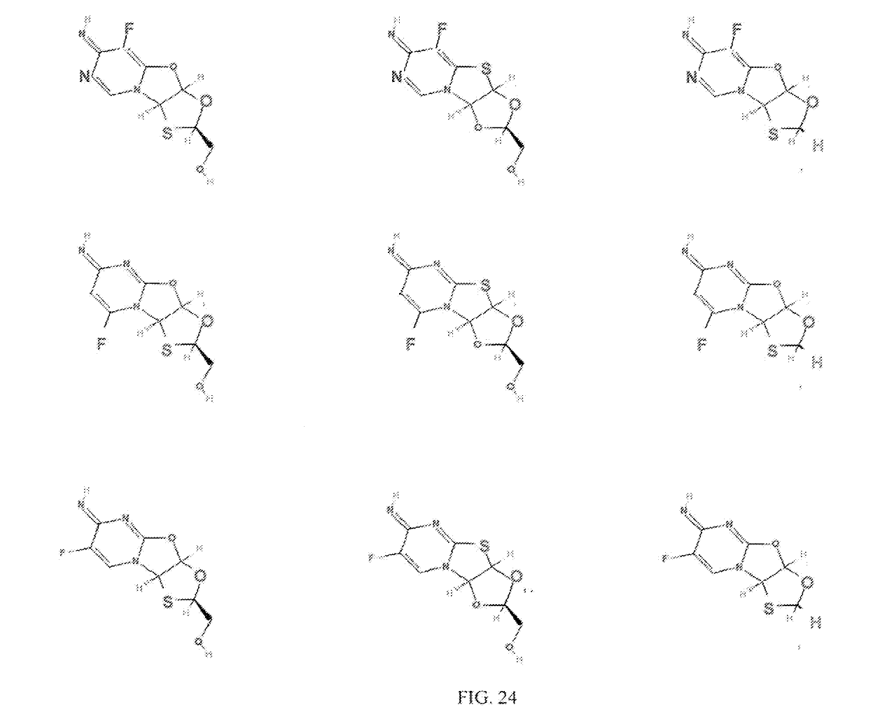

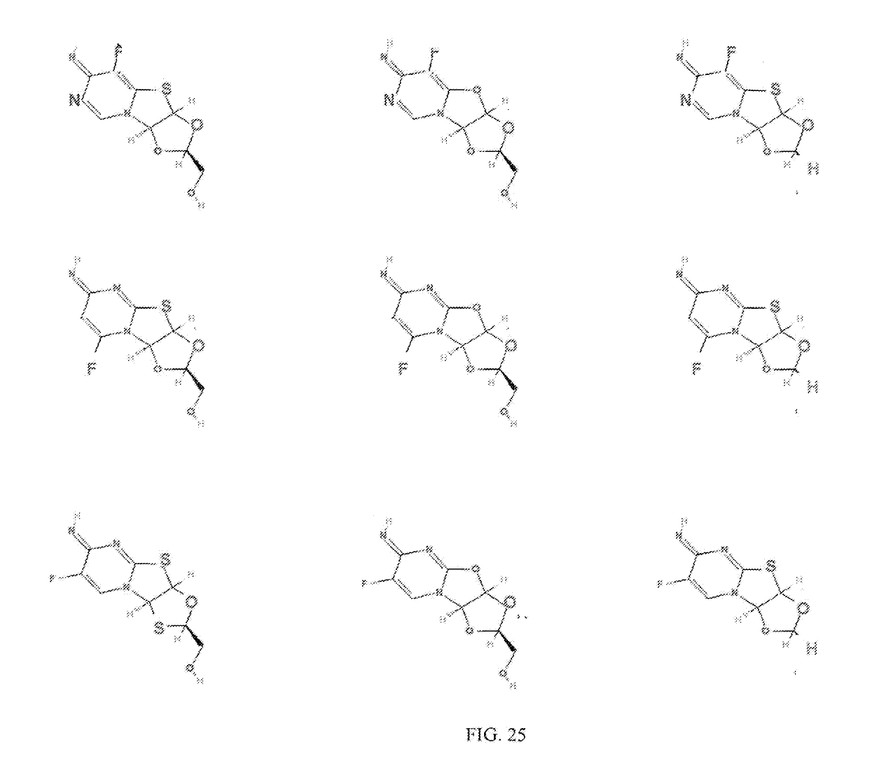

Embodiments relate to methods wherein the cyclocytidine analog may include a cyclocytidine. Embodiments include methods wherein the cyclopyrimidine analog may be shown in any of FIGS. 1-33.

Embodiments relate to methods of treating HIV infection with a cyclocytidine. According to embodiments, a process of treating HIV infection may include providing a cyclocytidine and administering the cyclocytidine to a patient infected with the HIV virus.

According to embodiments, a cyclocytidine may include pharmaceutically acceptable salts of O2,2′-cyclocytidine, O2,2′-cyclocytidine monoacetate (Cyclo-C, NSC: 129220, chemical formula: C9H10FN3O4.CIH), O2,2′-cyclocytidine hydrochloride, pharmaceutically acceptable salts of 5-fluoro-2,2′cyclocytidine and 5-fluoro-2,2′cyclocytidine (AAFC, NSC: 166641, chemical formula: C9H10FN3O4.CIH).

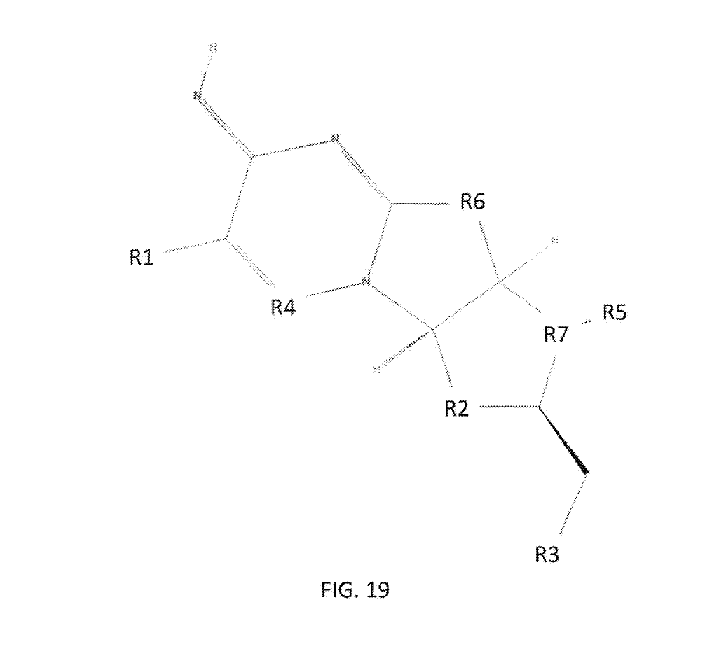

Referring to FIG. 19, a cyclocytidine may include a structure as shown in FIG. 19, where R1 can be any one of hydrogen, fluorine, chlorine, bromine or iodide; R2 can be any one of oxygen or sulfur; R3 can be any one of a hydroxyl group or hydrogen; R4 can be any one of hydrogen, fluorine, chlorine, or bromine; R5 can be any one of hydrogen, or a hydroxyl group; R6 can be any one of oxygen or sulfur; and R7 can be any one of carbon or oxygen.

Embodiments further relate to substituted cyclocytidines where one or more of R1, R2, R3 R4, R5, R6, and R7 may be substituted within an R group of similar size, valence, polarity, electron donating properties, or electron withdrawing properties, as the respective R groups set forth in paragraph 37 above.

Embodiments further relate to methods of treating animals, chordates, mammals, and humans infected with or at risk for infection with the HIV virus.

According to embodiments, cyclocytidine compounds can be administered in a cocktail comprising at least one cyclocytidine and between one and ninety-nine other antiretrovirals drugs. According to embodiments, cocktails may contain antiretroviral drugs from more than one class of antiretroviral drugs.

According to embodiments, antiretroviral drugs may include drugs that target various phases of the retrovirus-life-cycle. Antiretroviral drugs include nucleoside and nucleotide reverse transcriptase inhibitors (NRTI), non-nucleoside reverse transcriptase inhibitors, protease inhibitors, integrase inhibitors, entry inhibitors, and maturation inhibitors. Antiretroviral drugs include emtricitabine (an NRTI), tenofovir (an NRTI), efavirenz (a NNRTI), raltegravir (an integrase inhibitor), darunavir (a protease inhibitor), ritonavir (a protease inhibitor), atazanavir (a protease inhibitor), zidovudine, lamivudine, abacavir, lopinavir, stavudine, lamivudine, and nevirapine.

According to embodiments, antiretroviral drugs may include combivir (Glaxo Smith Kline), emtriva (Gilead sciences), epivir (GlaxoSmithKline), Epivir (Glaxo Smith Klein), Epzicom (Glaxo Smith Kline), Hivid (Hoffman-La Roche), Retrovir, Trizivir (Glaxo Smith Kline), lamivudine and zidovudine (GlaxoSmithKline), Emtricitabine (Gilead Sciences), lamivudine, 3TC (GlaxoSmithKline), abacavir/lamivudine (GlaxoSmithKline), zalcitabine, ddC, dideoxycytidine (Hoffmann-La Roche), zidovudine, AZT, azidothymidine, ZDV (Glaxo Smith Kline), abacavir, zidovudine, and lamivudine (Glaxo Smith Kline), tenofovir disoproxil/emtricitabine (Gilead Sciences, Inc.), enteric coated didanosine (Bristol Myers-Squibb), didanosine, ddI, dideoxyinosine (Bristol Myers-Squibb), tenofovir disoproxil fumarate (Gilead Sciences), stavudine, d4T (Bristol Myers-Squibb), and abacavir (Glaxo Smith Kline).

According to embodiments, nonnucleoside reverse transcriptase inhibitors (NNRTIs) may include delavirdine, DLV (Pfizer), efavirenz (Bristol Myers-Squibb), nevirapine (BI-RG-587, Boehringer Ingelheim).

According to embodiments, protease Inhibitors (PIs) may include amprenavir (GlaxoSmithKline), Tipranavir (Boehringer Ingelheim), saquinavir mesylate, SQV (Hoffmann-La Roche), lopinavir and ritonaviry (Abbott Laboratories), Fosamprenavir Calcium (GlaxoSmithKline), ritonavir, ABT-538 (Abbott Laboratories), darunavir (Tibotec, Inc.), atazanavir sulfate (Bristol-Myers Squibb), and nelfinavir mesylate, NFV (Agouron Pharmaceuticals).

According to embodiments, fusion inhibitors may include enfuvirtide, T-20 (Hoffmann-La Roche & Trimeris).

In embodiments, entry inhibitors may include maraviroc (Pfizer).

According to embodiments, HIV integrase strand-transfer inhibitors may include raltegravir (Merck & Co., Inc.).

According to embodiments anti-HIV treatment regimens may include combinations of drugs. According to embodiments multiclass combinations of drugs may include combivir (lamivudine and zidovudine, GlaxoSmithKline), emtriva (Emtricitabine and FTC, Gilead Sciences), epivir (lamivudine and 3TC, GlaxoSmithKline), epzicom (abacavir and lamivudine, GlaxoSmithKline), hivid (zalcitabine, dideoxycytidine, ddC (Hoffmann-La Roche), Retrovir (zidovudine, azidothymidine, AZT, ZDV (GlaxoSmithKline), Trizivir (abacavir, zidovudine, and lamivudine (GlaxoSmithKline), Truvada (tenofovir disoproxil fumarate and emtricitabine, Gilead Sciences, Inc.), videx EC (enteric coated didanosine, ddI EC, Bristol Myers-Squibb), videx (didanosine, dideoxyinosine, ddI, Bristol Myers-Squibb), viread (tenofovir disoproxil fumarate, TDF, Gilead), zerit (stavudine, d4T, Bristol Myers-Squibb), ziagen (abacavir sulfate, ABC (GlaxoSmithKline).

Embodiments relate to articles of manufacture wherein a cyclocytidine analog may include 5-fluoro-2,2′cyclocytidine (AAFC, NSC: 166641, chemical formula: C9H10FN3O4.CIH)). Embodiments also relate to methods wherein a cyclocytidine may include O2,2′-cyclocytidine monoacetate (Cyclo-C, NSC: 122940, chemical formula: C9H10FN3O4.CIH).

According to embodiments, compositions may be administered orally, parenterally, transdermally, bucally, nasally, mucosally, and sublingually or any combination thereof. As illustrated in one aspect of embodiments, compositions may include cyclocytidines, 5-fluoro-2,2′cyclocytidine, O2,2′-cyclocytidine, or analogs thereof.

According to embodiments, the process of administering a therapeutically effective amount may include administering an amount from about 1 ng to about 1000 mg per day. Embodiments relate to methods wherein said cyclocytidine analog may have an EC50 of less than about 500 nM. Embodiments relate to methods wherein said cyclocytidine may have an EC50 of less than about 500 nM. As illustrated in one aspect of embodiments, 5-fluorocyclocytidine may have an EC50 of about 700 μM. As illustrated in another aspect of embodiments, 5-fluorocyclocytidine may have an EC50 of about 10 nM.

According to embodiments, at least one process may include a step of selecting or identifying a patient in need of treatment. According to embodiments, a patient in need of treatment may be selected or identified as a patient presently infected with HIV or at presently at risk for infection with HIV. According to embodiments, patients presently infected could be identified with methods well known to skilled artisans including clinical methods, diagnostic methods, immunological detection methods, polymerase chain reaction (PCR) based methods, reverse transcriptase-PCR (RT-PCR) based methods, southern, northern or western analysis, physical examination, and/or radiological testing processes.

According to embodiments, an article of manufacture may include a kit may include a vessel containing at least one of O2,2′-Cyclocytidine, a pharmaceutically acceptable prodrug thereof and a salt thereof and at least one of 5-fluoro-2,2′-cyclocytidine, a pharmaceutically acceptable prodrug thereof and a salt thereof and an instruction to treat an infection of human immunodeficiency virus including administering to a patient an effective amount of the at least one of O2,2′-Cyclocytidine, a pharmaceutically acceptable prodrug thereof and a salt thereof and at least one of 5-fluoro-2,2′-cyclocytidine, a pharmaceutically acceptable prodrug thereof and a salt thereof. According to embodiments, an article of manufacture may optionally further contain at least one of a nucleoside reverse transcriptase inhibitor, a nucleotide reverse transcriptase inhibitor, a non-nucleoside reverse transcriptase inhibitors, a protease inhibitor, an integrase inhibitor, an entry inhibitor, a maturation inhibitor, tenofovir, raltegravir, darunavir, ritonavir, atazanavir, zidovudine, lamivudine, abacavir, lopinavir, stavudine, lamivudine, and nevirapine.

According to embodiments, an article of manufacture may further include a label that indicates that at least one of O2,2′-Cyclocytidine, a pharmaceutically acceptable prodrug thereof and a salt thereof and at least one of 5-fluoro-2,2′-cyclocytidine, a pharmaceutically acceptable prodrug thereof may be used with or without a nucleoside reverse transcriptase inhibitor, a nucleotide reverse transcriptase inhibitor, a non-nucleoside reverse transcriptase inhibitors, a protease inhibitor, an integrase inhibitor, an entry inhibitor, a maturation inhibitor, tenofovir, raltegravir, darunavir, ritonavir, atazanavir, zidovudine, lamivudine, abacavir, lopinavir, stavudine, lamivudine, and nevirapine for treating or ameliorating HIV infection.

Alternate embodiments may include a method of making a composition including recognizing that at least one of O2,2′-Cyclocytidine, a pharmaceutically acceptable prodrug thereof and a salt thereof and at least one of 5-fluoro-2,2′-cyclocytidine, a pharmaceutically acceptable prodrug thereof and a salt thereof have antiretroviral activity, and combining at least one of O2,2′-Cyclocytidine, a pharmaceutically acceptable prodrug thereof and a salt thereof and at least one of 5-fluoro-2,2′-cyclocytidine, a pharmaceutically acceptable prodrug thereof and a salt thereof with at least one of a nucleoside reverse transcriptase inhibitor, a nucleotide reverse transcriptase inhibitor, a non-nucleoside reverse transcriptase inhibitors, a protease inhibitor, an integrase inhibitor, an entry inhibitor, a maturation inhibitor, tenofovir, raltegravir, darunavir, ritonavir, atazanavir, zidovudine, lamivudine, abacavir, lopinavir, stavudine, lamivudine, and nevirapine.

According to embodiments, a composition may include a composition comprising one or more antiretroviral drugs and one or more of 5-fluoro-2,2′-cyclocytidine, O2,2′-Cyclocytidine, the compounds set forth in FIG. 19, and pharmaceutically acceptable prodrugs or salts salt thereof respectively.

According to embodiments, antiretroviral drugs may include one or more of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor.

According to embodiments, antiretroviral drugs may include one or more of lamivudine, tenofovir, raltegravir, darunavir, ritonavir, atazanavir, zidovudine, abacavir, lopinavir, stavudine, and nevirapine.

According to embodiments, compositions may include about 2 to about 99 antiretroviral drugs.

Additional embodiments may include methods of combining which may include one or more of mixing, oral administration, parenteral administration, transdermal administration, buccal administration, nasal administration, mucosal administration, and sublingual administration.

Still additional embodiments may include a process to treat an infection of human immunodeficiency virus (HIV) comprising administering to a patient a therapeutically effective amount of a first compound wherein the first compound may be at least one of O2,2′-Cyclocytidine (CycloC), the compounds set forth in FIG. 19, and pharmaceutically acceptable prodrugs or salts salt thereof respectively.

According to additional embodiments, processes to treat an infection of human immunodeficiency virus may include administering to the patient a therapeutically effective amount of at least one second compound wherein said at least one second compound may be selected from the group consisting of 5-fluoro-2,2′-cyclocytidine, a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, a maturation inhibitor, tenofovir, raltegravir, darunavir, ritonavir, atazanavir, zidovudine, lamivudine, abacavir, lopinavir, stavudine, lamivudine, and nevirapine.

According to embodiments, patients may include patients that are mammals. An additional aspect of an embodiment includes a patient wherein the patient may include a patient with a history of recent contact with a new sex partner. Additional aspects of embodiments relate to patients who may have begun salvage therapy.

Aspects of embodiments relate to routes of administration of compounds including wherein the compound may be administered at least one of orally, parenterally, transdermally, bucally, nasally, mucosally, and sublingually. In a further aspect of an embodiment, processes may include selecting a patient in need of such treatment.

Aspects of embodiments relate to processes wherein the therapeutically effective amount may be from about 1 ng to about 1000 mg per day.

In still another aspect of embodiment, a process to treat an infection of human immunodeficiency virus may include administering to a patient a therapeutically effective amount of at least one of 5-fluoro-2,2′-cyclocytidine, the compounds set forth in FIG. 19, and pharmaceutically acceptable prodrugs or salts salt thereof respectively.

In still another aspect of embodiment, a process for administering to the patient a therapeutically effective amount of at least one second compound may include wherein said at least one second compound may be selected from the group consisting of lamivudine, 5-fluoro-2,2′-cyclocytidine, a nucleoside reverse transcriptase inhibitor, a nucleotide reverse transcriptase inhibitor, a non-nucleoside reverse transcriptase inhibitors, a protease inhibitor, an integrase inhibitor, an entry inhibitor, a maturation inhibitor, tenofovir, raltegravir, darunavir, ritonavir, atazanavir, zidovudine, abacavir, lopinavir, stavudine, and nevirapine.

In still another aspect of embodiment, processes may include administering at least one of the at least one of 5-fluoro-2,2′-cyclocytidine, pharmaceutically acceptable prodrug thereof and salt thereof wherein the administering may include at least one of orally, parenterally, transdermally, bucally, nasally, mucosally, and sublingually.

Aspects of embodiments may include a method of treating HIV infection, which may include identifying a patient in need of such treatment. Additional aspects of embodiments may include a method of handpicking a patient in need of such treatment. In yet another aspect of an embodiment, selecting may include identifying.

According to aspects of embodiments, the therapeutically effective amount may be from about 1 ng to about 1000 mg per day.

Additional embodiments may include a method of making a composition which may comprise combining one or more antiretroviral drugs with at least one of O2,2′-Cyclocytidine, the compounds set forth in FIG. 19, 5-fluoro-2,2′-cyclocytidine, and pharmaceutically acceptable prodrugs or salts salt thereof respectively.

In still another aspect of an embodiment, one or more antiretroviral drugs may comprise one or more of lamivudine, tenofovir, raltegravir, darunavir, ritonavir, atazanavir, zidovudine, abacavir, lopinavir, stavudine, and nevirapine.

In a further aspect of an embodiment combining may include combining by one or more of mixing, ingestion, oral administration, parenteral administration, transdermal administration, buccal administration, nasal administration, mucosal administration, and sublingual administration.

Aspects of embodiments include a method of treating HIV infection in patients who begun or failed salvage pathway therapy. According to embodiments, salvage pathway therapy is when an HIV patient has failed a previous anti-retroviral treatment regimen.

According to embodiments, one or more of O2,2′-Cyclocytidine, the compounds set forth in FIG. 19, 5-fluoro-2,2′-cyclocytidine, and pharmaceutically acceptable prodrugs or salts salt thereof respectively maybe combined with one or more of Atripla (Efavirenz/Emtricitabine/Tenofovir disoproxil fumarate), Combivir (Lamivudine/Zidovudine), Complera (Emtricitabine/Rilpivirine/Tenofovir disoproxil fumarate), Epzicom (Abacavir/Lamivudine), Trizivir (Abacavir/Lamivudine/Zidovudine), and Truvada (Emtricitabine/Tenofovir disoproxil fumarate).

According to aspects of embodiments, treatment regimens may include administering to a patient a therapeutically effective amount of one or more of O2,2′-Cyclocytidine, a pharmaceutically acceptable prodrug thereof and a salt thereof, wherein said therapeutically effective amount minimizes HIV viral load.

According to aspects of embodiments, treatment regimens may include administering to a patient a therapeutically effective amount of one or more of 5-fluoro-2,2′-cyclocytidine, a pharmaceutically acceptable prodrug thereof and a salt thereof, wherein said therapeutically effective amount minimizes HIV viral load.

Still additional aspects of embodiments include methods of contacting one or more cells infected or at risk for infection with the HIV virus with an active compound or composition as set forth herein. Cells may include in vitro, ex vivo, and in vivo cells, as well as cells in a body.

Still additional aspects of embodiments include methods wherein a cell may be contacted with at least one of O2,2′-Cyclocytidine (CycloC), the compounds set forth in FIG. 19, and pharmaceutically acceptable prodrugs or salts salt thereof respectively.

Aspects of embodiments include constellations of both treatment and prevention methods.

Deoxycytidine kinase is an enzyme in the dNTP salvage pathway and has a role in lymphocyte maturation and immune system function. Lymphocytes are dependent on the salvage pathway for supplies of dNTPs for DNA replication and cell cycle progression. Reliance on the dNTP salvage pathway for dNTP requirements leaves lymphocytes vulnerable to dNTP pool imbalances and dNTP related metabolic stress.

Resting lymphocytes depend on the dNTP salvage pathway for 70% of dTTP requirements. This occurs through a series of salvage reactions starting with the conversion of dCyt to dCMP via deoxycytidine kinase followed by the conversion of dCMP to dUMP via cytidylate deaminase and then the conversion of dUMP to dTMP via thymidine synthetase (TS).

In contrast, activated lymphocytes experience a rapid rise in deoxycytidine kinase (30%+) and thymidine kinase I (100%+) activity. Thymidine kinase converts deoxyuridine monophosphate (dUrd) directly into dUMP the precursor of dTMP and deoxythymidine (dTh) into dTMP.

Lymphocyte activation leads to a rapid rise in dTTP levels via thymidylate kinase I (100%+) activity. Increased deoxycytidine kinase activity would normally bring the pools of deoxyadenosine triphosphate (dATP), deoxycytidine triphosphate (dCTP) and deoxyguanosine triphosphate (dGTP) into balance. If deoxycytidine activity is inhibited an imbalance of deoxythymidine triphosphate (dTTP) pools may build leading to initiation of survival strategies.

dTTP is an allosteric inhibitor of ribonulceotide reductase, an enzyme in the de novo dNTP pathway further antagonizing the cells ability to re-balance dNTP pools.

Resting lymphocytes due to the low levels of deoxycytidine kinase (<15%) and thymidilate kinase I (<1%) activity, are less susceptible to dNTP pool imbalances brought on by the competitive inhibition of deoxycytidine kinase.

The therapeutic activity (anti-cancer, anti-viral, immunomodulation) of AAFC and related cyclocytidine analogs may be competitive inhibition of deoxycytidine kinase.

AAFC and related cyclocytidine analogs do not appear to be pro-drugs, are resistant to cellular phosphorylation, and deaminase activity enhancing therapeutic value.

AAFC related cyclocytidine analogs or deaminated by products might inhibit or be substrates for other cellular enzymes leading to an anti-HIV effect. Phosphorylated AAFC and related cyclocytidine analogs may have mutagenic qualities that could interfere with reverse transcriptase similar to the action of Acyclovir in herpes therapy.

The inhibition of deoxycytidine kinase by AAFC and related cyclocytidine analogs combined with the dependence of lymphocytes on the dNTP salvage pathway leads to a dNTP pool imbalance.

T-lymphocytes of both asymptomatic and symptomatic HIV+ individuals exhibit anergetic qualities, nucleotide imbalances and a resistance to the uptake of thymidine, indicating a dNTP nucleotide pool imbalance specifically in deoxythymidine and related phospho derivatives.

Late stage HIV infection induces a similar state brought on by the inhibition of deoxycytidine kinase by AAFC and related cyclocytidine analogs in treated and activated T lymphocytes.

The correct balance of the dNTP pool has a role in efficient replication of DNA by either DNA or RNA polymerases. A dNTP pool imbalance leads to mis-match dNTP incorporations and lethal mutations during replication.

T lymphocytes treated with AAFC and related cyclocytidine analogs that are exposed to HIV infection and activation undergo a rapid rise in dTTP pools leading to lethal mutations in viral replication mediated by reverse transcriptase halting the viral life cycle.

Post exposure of HIV+ cells to AAFC and related cyclocytidine analogs causes a dNTP pool imbalance in activated cells, specifically dTTP which may lead to the induction of survival strategies and phosphothymine induced cytotoxicity.

The inhibition of deoxycytidine kinase activity affects the bioavailability of phosphocytidine derivatives and the expansion and maintenance of the cell membrane.

HIV+ lymphocytes treated with AAFC and related cyclocytidine analogs experience a combination of; induced lethal mutations during reverse transcription, the additive effect of thymine-induced cytotoxicity brought on by the activation of the infected lymphocyte by HIV viral attachment proteins and a reduction of phospholipid metabolism.

AAFC and related cyclocytidine analogs have therapeutic value in the treatment of other viral and bacterial infections.

Given the effect of the activation of lymphocytes by the inhibition of deoxycytidine kinase, AAFC and related cyclocytidine analogs may have therapeutic value in medical situations where the depression of the lymphocytic response would provide efficacy, such as in organ transplant rejection, arthritis and other autoimmune diseases.

Research shows that deoxycytidine (dCyt) is capable of reversing the effects of deoxycytidine kinase inhibition by AAFC and related cyclocytidine analogs. Addition of dCyt overcomes the competitive inhibition of deoxycytidine kinase and relives the dNTP pool imbalance permitting the return of proper cellular functions in the immune response. dCyt therapy may be an effective therapeutic in relaxing NTP and dNTP pool imbalances observed in asymptomatic and symptomatic HIV+ individuals.

Screening hundreds of compounds with an HIV reporter cell line led to the discovery of a new use for known and clinically trialed anti-cancer reagents that surprisingly exhibited potent anti-HIV activity in vitro. One particular reagent AAFC, significantly inhibited HIV replication in experimental T cell cancer lines (EC50<500 nM) and in primary peripheral blood mononuclear cells (EC50<700 pM). Past and present research indicates that this activity is a direct result of the competitive inhibition of enzymes—specifically deoxycytidine kinase (dCK) of the dNTP salvage pathway leading to imbalances in cytosolic deoxynucleotide pools causing lethal mutations during reverse transcription and the induction of HIV mediated cytotoxicity via the additive antagonism of dNTP pools, specifically dTTP.

The purine and pyrimidine nucleotide de novo and salvage pathways are regulators of lymphocytic maturation and immune response. T lymphocytes which develop abnormal nucleotide and deoxynucleotide pools show markedly differentiated responses to stimulus leading to impairment of immune system function.

HIV infected T-lymphocytes exhibit abnormal purine and pyrimidine ribonucleotide pools which lead to anergetic qualities such as impaired response to stimulus and apoptosis. Abnormalities in cytidine triphosphate (CTP), uridine diphosphate (UDP) and uridine triphosphate (UTP) bioavailability following stimulation and the inability of HIV+ T-lymphocytes to incorporate tritiated thymidine point to a significant disruption in both the purine and pyrimidine salvage dNTP and NTP pathways. The mechanism by which HIV causes this phenomenon is still unknown, but may point to the inhibition of enzymes involved with the regulation of nucleotide pools.

Cancer, Cancer Therapy and Nucleotide Metabolism

An enzyme involved in T cell maturation and function is the dNTP salvage enzyme deoxycytidine kinase (dCK) which is found in high levels in the thymus and other lymphoid tissues and organs. dCK plays a role in the pyrimidine salvage pathway and is regulated via feedback inhibition by components and downstream products of the pyrimidine de novo and salvage pathways.

Defects in the activity of enzymes involved in nucleotide metabolism include cytidine kinases having a role in cancer pathogenesis. Cancer cells exhibit abnormalities and mutations in the enzymes involved with the regulation of nucleotide metabolism: this condition may promote rapid growth and division. Therapies that target the inhibition of the de novo and salvage nucleotide pathways or have a role for it for prodrug activation have been the basis of numerous anti-cancer and anti-viral therapies. Many of these reagents are nucleoside analogs that become phosphorylated by dexyocytidine kinase in order to exert their effect via the inhibition of DNA/RNA polymerases and ribonucleotide reductase (RNR).

The relationship between cancer and retrovirus may be the direct result of retrovirus infection. This relationship led to interest in cancer treatments and therapies—including interest in Ara-C42. Ara-C is an antimetabolite that is given intravenously because of its rapid conversion into its active form cytosine arabinoside triphosphate by the cellular enzyme deoxycytidine kinase and its deamination by cellular enzymes such as cytidine deaminase that convert it further into inactive or active by products. Once activated, cytarabine may be a potent inhibitor of DNA polymerase and ribonucleotide reductases having roles in DNA synthesis.

The effectiveness of Ara-C in cancer treatment led to interest in analogs of the drug that may be as effective and slowly released in the body without significant side effects. One such analog was AAFC or 5-fluorocyclocytidine. AAFC was found to possess anticancer efficacy but not on the same level of Ara-C. Metabolic studies confirmed that AAFC is less easily phosphorylated as compared to Ara-C and that over 80% of the reagent passed through the body unchanged. Due to AAFC's apparent lack of cancer efficacy in some types of cancer during Phase I and II clinical trials in comparison to Ara-C and its low conversion into the active form of Ara-C, interest in the compound lapsed.

Early research on AAFC showed that the compound inhibited thymidine incorporation into the acid-soluble fraction (DNA) of the cells. Cell growth inhibition by AAFC was reversed by deoxycytidine but not by thymidine and deoxyuridine. AAFC's direct method of action may be the competitive inhibition of the dNTP salvage enzyme deoxcytidine kinase (dCK) and the formation of dNTPs specifically dGMP, dCMP and dAMP.

The competitive inhibition of dCK activity may lead to a disruption in the bioavailability of the 5′-phosphorylation of deoxynucleosides deoxycytidine (dCTP), deoxyguanosine (dGTP) and deoxyadenosine (dATP) leading to reduction in these vital dNTPs for DNA synthesis. Cancer cell lines having increased requirements for dNTPs needed for rapid growth and proliferation may be vulnerable to the down regulation of dCK without a corresponding increase dNTP production via the de novo pathway. Some cancer cell lines develop drug resistance on this basis. In comparison, resting or inactive lymphocytes have very low requirements for dNTPs.

Deoxynucleotide (dNTP) pools are produced by either the direct conversion of CTP, UTP, GTP and ATP by ribonucleotide reductase (RR) into deoxy forms or the salvage of previously utilized nucleotides into dNTPs via dCK phosphorylation or via the direct conversion of dCMP to dUMP via cytidylate deaminase (CDA) and then the conversion of dUMP into dTMP catalyzed by thymidine synthetase. Studies show that T cells possess a unique quality in that they rely on the dNTP salvage pathway instead of the de novo pathway for the availability of dNTPs. This leaves T lymphocytes in particular vulnerable to dNTP manipulation via either the inhibition/upregulation of salvage enzymes such as dCK or cytidine deaminase enzymes (CDA).

The reliance on the salvage pathway and the enzymes dCK and CDA for metabolic supplies of dNTPs is unique to lymphocytes. This may be linked to the need for dCTP in the production of liponucletides which are then converted to phospholipids and used in membrane growth and maintenance or directly to the T cell negative selection process in the thymus. Resting lymphocytes rely on the cytidylate deaminase salvage pathway to convert cCMP into dUMP which is then converted by thymidilate synthetase (TS) to dTTP Research indicates that resting lymphocytes convert up to 70% of added dCYT directly into dTTP via the CDA and TS salvage pathway. The reliance on the salvage pathway for the regulation of proper dNTP pools optimal for DNA synthesis and metabolic pathways leaves activated lymphocytes vulnerable to a dCK bottleneck.

Thus, one can draw a line between the reduction in dCTP levels in T-lymphocytes via the inhibition of dCK leading to an increase in dTMP, dTDP and dTTP levels. dCTP is an active inhibitor of dCK and therefore it regulates the production of dNTPs in the salvage pathway. Scientific observations show that once activated T lymphocytes show a 100% increase in the activity of thymidilate kinase I (TKI) and a 30% rise in dCK activity over relatively low levels in resting PBMCs. This means that once activated T lymphocytes will experience a significant rise in TKI activity leading to a deoxythymidine pool imbalance and a high rate of thymine-induced cytotoxicity dependent on the active state of the cell.

Elevation of thymidine (dTHD) levels may inhibit the growth of most mammalian cells. dTHD toxicity may be due to an increase of the dTTP pool. In addition, dTTP levels may antagonize the nucleotide pool imbalance by allosterically inhibiting ribonucleotide reductase leading to significant disruption in DNA synthesis. This explains the inability of T cells to utilize thymidine in numerous studies via dCK inhibition.

AAFC and Cyclocytidine Derivatives

There are differences between Ara-C and AAFC. Like many HIV nucleoside reverse transcriptase inhibitors such as AZT, Ara-C is a prodrug that may be phosphorylated into its active form Ara-CTP by dCK in order to inhibit DNA polymerase. Cells resistant to Ara-C either have dCK mutations preventing the formation of Ara-CTP or upregulate RNR or CDA activity in order to balance dNTP pools.

The chemical structure of AAFC contains a cyclo-bond that is not readily phosphorylated as shown in animal and human trials, but rather in the case of our research does not require activation. Without being limited to any particular theory, applicants believe the mechanism of action is the direct competitive inhibition of deoxycytidine kinase and phosphorylation or deamination to Ara-C may deactivate its mechanism of action.

AAFC is not readily converted into the highly active cancer agent AraF-CTP. As a competitive inhibitor of dCK, its effects would be transient and readily overcome by cancer cells via upregulation of dCK, RNR or CDA. This may make AAFC effective against some cancer types, but ineffective against others. In contrast, the effectiveness of Ara-C in cancer therapy is the direct inhibition of DNA polymerase and cytotoxicty.

Without being limited to any particular theory, the effects of AAFC on dCTP levels may be reversed by the addition of deoxycytidine supporting the idea that AAFC may competitively inhibits dCK and the formation of dGMP. dAMP and dCMP and may not directly inhibit DNA polymerase as in the case of Ara-C.

NRTIs and AAFC

The entire class of nucleotide reverse transcriptase inhibitors (NRTIs) which includes Zidovudine (AZT) the first drug approved for anti-retroviral therapy, are deoxynucleoside derivatives that contain a variety of different chemical modalities at the 3′ position, and number of ribose substituted carbon or oxygen groups and in general an alcohol group at the 6′ position. This alcohol group may be phosphorylated by deoxycytidine kinase into its active form. NRTI's do not inhibit reverse transcriptase in the pro-drug form and normally may be incubated in vitro up to 24 hours pre-exposure to HIV in order become activated by phosphorylation. Once activated the drugs mechanism of action is the competitive inhibition of reverse transcription of viral RNA/DNA via chain termination due to the lack of an alcohol (OH) group at the 3′ of the ribose ring. Resistance to NRTIs is either a direct result of viral mutation, loss of dCK activity or the selection of cells that have upregulated salvage or de novo nucleotide pathways.

AAFC Has Differences to the NRTI Class of Pro-Drugs

AAFC contains the alcohol group at the 3′ position that is lacking in NRTIs and is directly related to the mechanism of action of this class of drug. AAFC contains a cyclo-bond between the ribose and pyrimidine rings effectively forming a three-ring motif and appears related to its stability. AAFC may not be readily phosphorylated in vitro or in vivo. AAFC is effective in vitro at inhibiting HIV replication post infection exposure to HIV-1.

AAFC is effective in vitro at directly inhibiting HIV replication at about <500 nM concentrations in a number of experimental cell lines and in primary cells. Unlike prodrug NRTIs that benefit from pre-exposure activation via phosphorylation, research indicates that even post exposure of cells infected with HIV-I to AAFC can dramatically inhibit HIV replication in experimental cell lines.

AAFCs mechanism of action may be resistance to phosphorylation and deaminase activity and the basal competitive inhibition of dCK.

Research shows that T cells are reliant on the salvage pathway for their dNTP requirements. Therefore, T cells are particularly susceptible to dNTP manipulation, research points to the idea that this is a pathogenic strategy of HIV.

Competitive inhibition of dCK (salvage dNTP enzyme) may result in a drop in the levels of dCTP, dATP and dGTP and a rise in dTTP which is produced by thymidilate kinase in activated T lymphocytes.

dTTP allosterically may inhibit RNR which is the de novo pathway for the production of dNTPs from NTPs, further skewing the dNTP pool imbalance.

In resting T cells the levels of dCK and TKI activity is low in comparison to the activated state and observations indicate that these enzymes may be activated via phosphorylation. Studies indicate that HIV infection stimulates cDA and TKI activity which may lead to dTTP induced toxicity.

Predominance of dTTP may lead to metabolic stress, a drop in global gene expression and metabolism. Research shows that both AAFC treated T cells and primary T cells from both asymptomatic and symptomatic HIV+ patients are unable to uptake thymidine.

The effects of AAFC may be reversed by the addition of deoxycytidine to the cell culture resulting in the normalization of dNTP pools supporting the idea that AAFC may competitively inhibit cDK and that its effects may be reversible.

AAFC is a strong inhibitor of HIV replication in vitro. The dependence of the immune system on dCK activity is well documented. In this light competitive inhibition and correct levels of AAFC may be fine-tuned to permit efficacy against HIV while permitting suitable levels of immuno-responsiveness.

AAFC may be a competitive inhibitor due to the molecules stability and its resistance to cellular kinase phosphorylation. Research indicates that phosphorylation may deactivate its activity and effects on the HIV life cycle as indicated by analog HIV assays.

AAFC's mechanism of action may be the competitive inhibition of (dCK) that has a direct impact on the HIV life cycle in lymphocytes. Resting T lymphocytes as compared to activated, have low dNTP requirements and it appears that the inhibitory effect of AAFC on dCK is transient or mild. As a competitive inhibitor, AAFC may antagonize dNTP production and it is likely that the resting lymphocytes can mediate dNTP pools effectively under low stress conditions even while dNTP production is abated.

In contrast, activated lymphocytes experience a rapid rise in TKI activity (100%) leading to significant increases in dTTP. The inhibition of dCK under activated conditions produces an untenable dNTP pool imbalance leading to thymine-induced cytotoxicity.

The prime target for HIV attachment/entry proteins such as GPI60 are the T cell receptors CXCR4 and CCR5. The attachment, entry and activation of the HIV viral life cycle are directly correlated with the activation of lymphocytes. Although HIV does appear to enter resting T cells, it is the activation of lymphocytes that initiates the production of viral particles. Our experiments indicate that if dCK is competitively inhibited then the rapid rise in dTTP pools caused by the activation of lymphocytes by viral entry and replication may not be mediated and the cells experience thymine induced cytotoxicity.

### EXAMPLE EMBODIMENTS

### Example 1

### Screening Compounds for Anti-HIV Activity

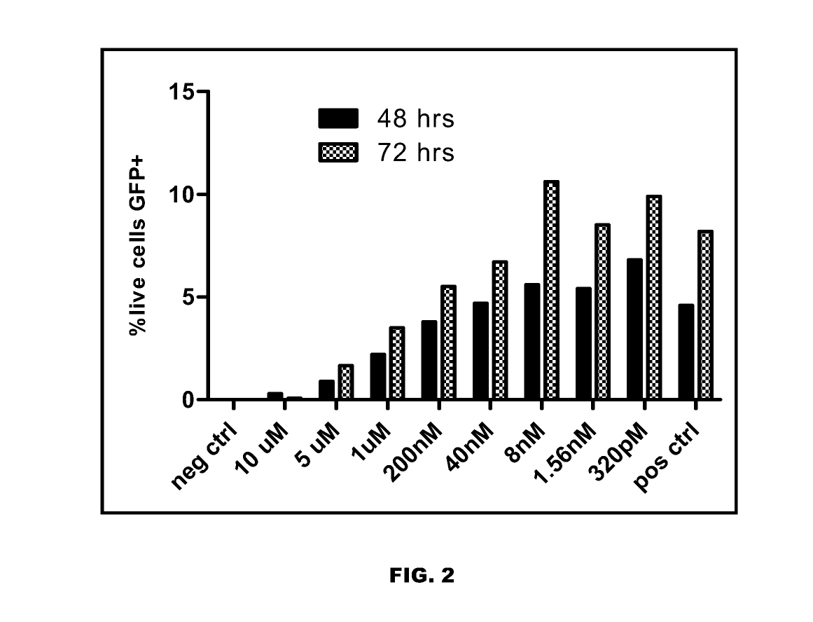

In order to identify compounds with anti-retroviral activity, 1,364 reagents were screened. 96 well plates containing the DTP/NIH diversity set were obtained from the National Institutes of Health (NIH) located in Frederick Md. The agents contained within the 96 well plates were pre-suspended in 10 mM DMSO. The Vsvg pseudotyped virus stock was cultivated according to protocol. Clone 69 reporter primary T cells were prepared. Five million clone 69 cells were suspended in 25 ml RPMI (Roswell Park Memorial Institute)-+10% FBS (Fetal Buffered Saline). One μL of reagent from the DTP/NIH test plate was transferred to and contacted with the resuspended cells. 20 μL of frozen and thawed Vsvg virus stock was then added to each well. The plate was then incubated for 48 hours at 37° C. 10 μL of 10× lysis buffer was dispensed into the luciferase reading plate. 200 μL of sample were transferred into the lysis buffer in the test plate and were marked. Plates were covered with foil and incubated at room temperature on the plate rocker 20 minutes. Plates were read on a Promega GloMax® 96 plate reader using 30 μL of 10× luciferase buffer with a 12 second delay and a 1 second integration time. Clone 64 luciferase reporter CEM-ss cells (50K) were seeded (RPMI+10% FBS) in 96 well plates, incubated for one hour with 20 uM of test reagents and infected with 20 ul (20 ng) of vsv-g pseudotyped HIV-1 virus. Samples were analyzed via luminometer at 48 hours post infection. (See FIG. 1). Reagents that inhibited luciferase expression below 1000 lumens were retested to confirm activity. (See FIG. 2-4).

G11 HIV reporter cells (200K) were seeded in 5 ml falcon tubes (RPMI+10% FBS) and incubated with the indicated amounts of NSC129220 (CAS 10212-28-9) for one hour before infection with HIV-1 (wt) virus (250 ng). Samples were stained with propidium iodide and analyzed for GFP expression via flow cytometer at 48 and 72 hours post infection. (See FIG. 2).

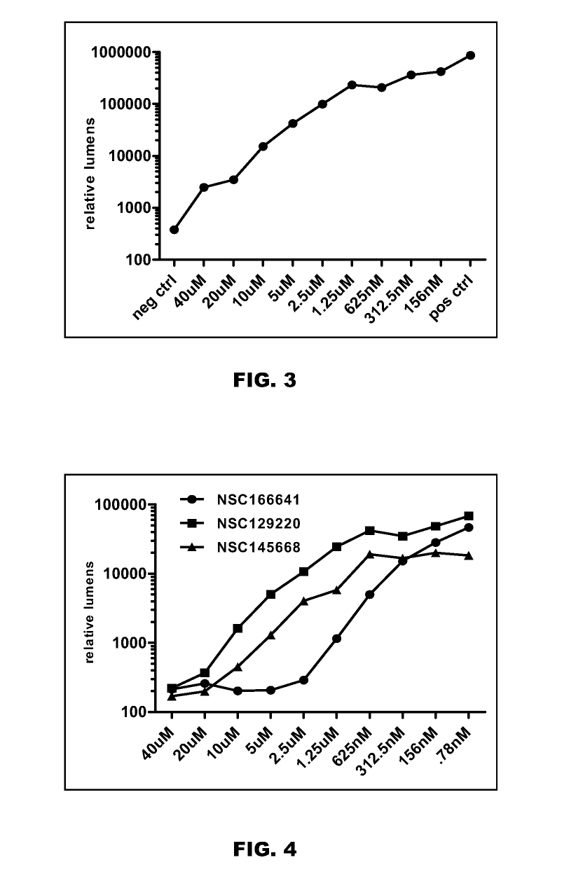

Clone 64 luciferase reporter CEM-ss cells (50K) were seeded (RPMI+10% FBS in 96 well plates and incubated for one hour with the indicated amount of NSC 1229220 before infection with 20 ul (20 ng) of vsv-g pseudotyped HIV-1 virus. Results were analyzed via luminometer 48 hours post infection. (See FIG. 3).

### Example 2

### Screening 5-Fluoro-2,2′-cyclocytidine, O2,2′-cyclocytidine monoacetate, and O2,2′-cyclocytidine hydrochloride for Anti-Retroviral Activity

5-Fluoro-2,2′-cyclocytidine, O2,2′-cyclocytidine monoacetate, and O2,2′-cyclocytidine hydrochloride were screened as set forth in Example 1. Clone 64 Luciferase reporter CEM-ss cells (50K) were seeded (RPMI+10% FBS) in 96 well plates incubated for one hour with the indicated reagents before infection with 20 ul (20 ng) of vsv-g pseudotyped HIV-1 virus. Results were analyzed via luminometer 48 hours post infection. (See FIG. 4).

Anti-HIV activity was detected at low levels of 5-Fluoro-2,2′-cyclocytidine, O2,2′-cyclocytidine monoacetate, and O2,2′-cyclocytidine hydrochloride. Activity was seen at doses as low as 40 μM in 0.4% DMSO. (See FIGS. 1-4, particularly FIG. 4).

### Example 3

### Screening Ethoxyphenol (NSC 125531), Hydroxyphenol (NSC 98938), Benzodiazepen (NSC 66020), Propanedioate (NSC 126224), and Methoxyellipticine (NSC 69187) for Anti-Retroviral Activity

Ethoxyphenol, hydroxyphenol, benzodiazipen, propanedioate, methoxyellipticine were screened as set forth in Example 1. Anti-HIV activity was detected with ethoxyphenol, hydroxyphenol, benzodiazipen, propanedioate, methoxyellipticine. Activity was not detected. See FIG. 1.

### Example 4

### Screening Compounds for Anti-Retroviral Activity

Benzoic acid, napthalen, octahydrophenthridine, dihydropyridine, and ethyl carbamate were among the 1,364 reagents screened as set forth in Example 1. Activity was not detected with Benzoic acid, napthalen, octahydrophenthridine, dihydropyridine, or ethyl carbamate. See FIGS. 1-4.

### Example 5

### Flori and O2,2′-Cyclocytidine Monoacetate at Various Concentrations

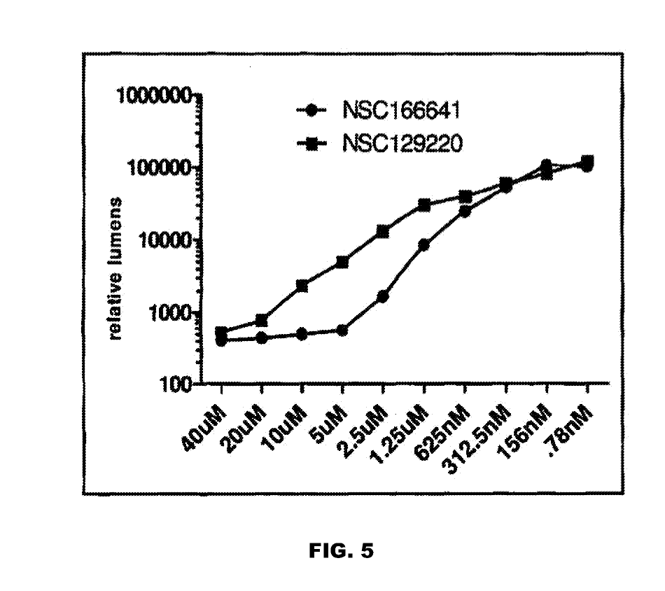

Clone 64 luciferase reporter CEM-ss cells (50K) were seeded (RPMI+10% FBS) in 96 well plates incubated for one hour with the indicated concentration of NSC166641 and NSC 129220 before infection with 20 ul of vsv-g pseudotyped HIV-1 virus. Results were analyzed via luminometer at 48 hours post infection. Dose-dependent activity was detected. (See FIG. 5).

### Example 6

### Flori at Various Concentrations

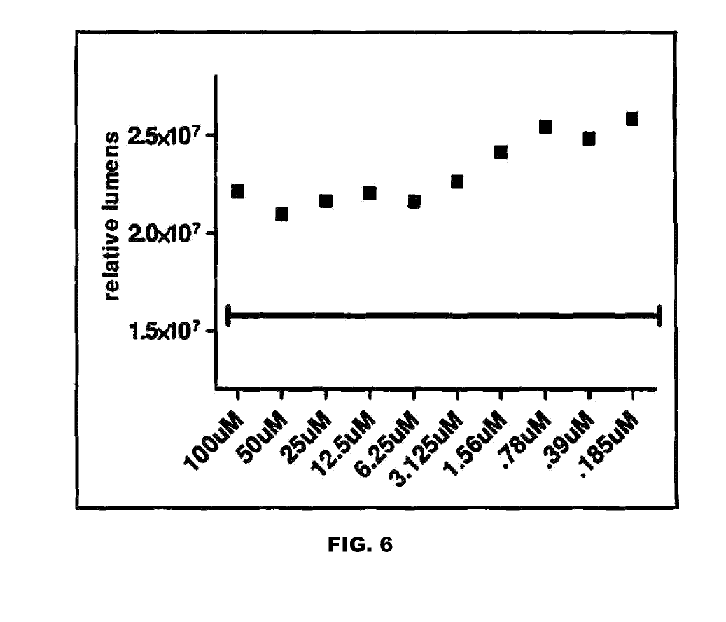

CEM-ss cells were plated (RPMI+10% FBS) with the indicated amounts of NSC 166641 for 48 hours and then tested for cell growth via the Promega CellTiter-Glo®Luminescent Cell Viability Assay system. The bar indicates 50% cell growth inhibition. (See FIG. 6).

### Example 7

### Dose Response Effects of 5-fluoro-2,2′cyclocytidine (AAFC) on HIV-1 Infected Cells

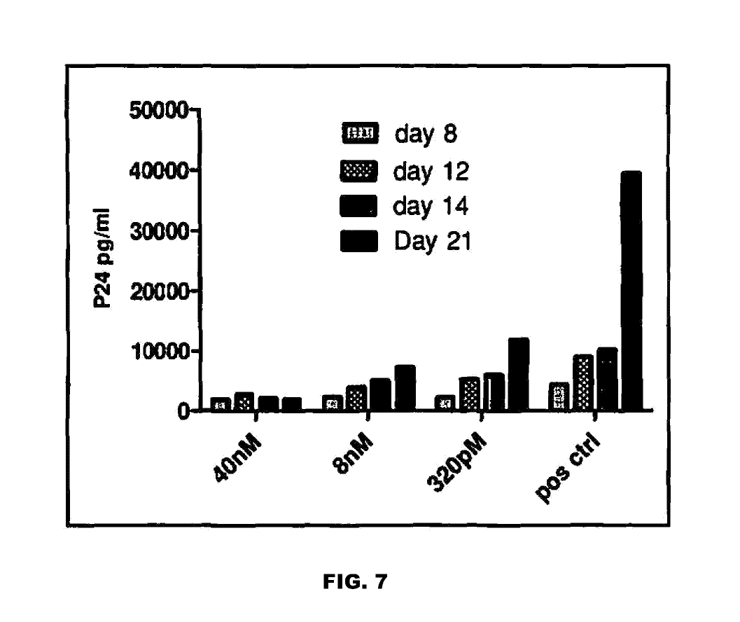

Human peripheral blood mononuclear cells (PBMCs) purified from donors were cultured (RPMI+10% FBS) and stimulated pre-infection (24 hrs) and every 3 days for three weeks with 2 ng/ml of phytoheaemagglutinin (PHA) and 1 U/ml IL-2. 24 hours following the initial stimulation the samples were incubated for one hour with the indicated amounts of NSC166641 and then infected with 300 ng HIV-1 (wt). Samples were analyzed via ELISA p24. (See FIG. 7).

### Example 8

### Effects of Flori on Total HIV-1 (Wt) DNA Copy Number

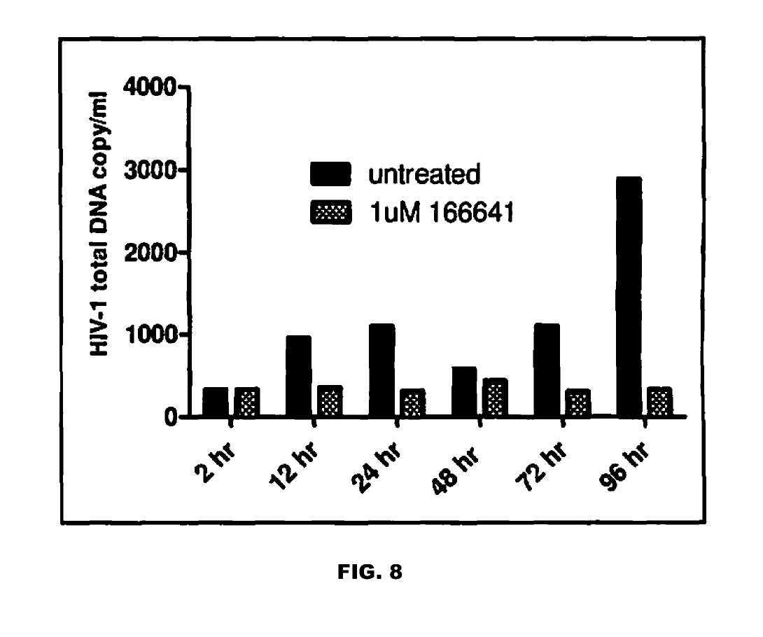

NSC166641 (1 uM) treated and untreated CEM. ss cells (2×108, were incubated with reagent 1 hour before infection with 1500 ng HIV-1 (wt)+bezonase. Samples were harvested at the specified times and analyzed via RT-PCR. (See FIG. 8).

### Example 9

### Flori at Various Concentrations

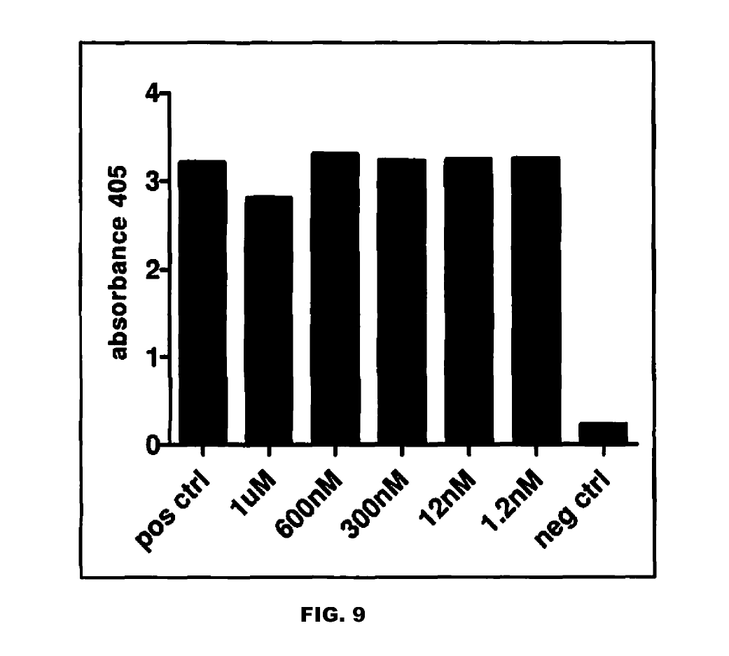

Results of the Roche reverse transcriptase (AT) assay kit utilizing the indicated amounts of NSC166641. RT activity was measured as a percentage compared to the positive control. Results show little or no inhibition of RT. (See FIG. 9).

### Example 10

### Cellular Growth Inhibition

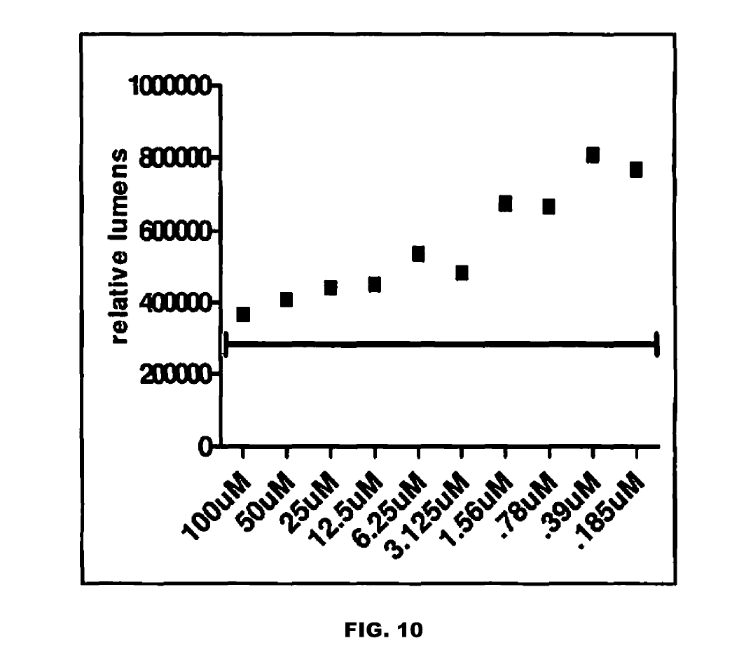

PBMCs purified from donor (x76) were plated (RPMI+10% FBS) with the indicated amounts of NSC166641 for 48 hours and then tested for cell growth inhibition via the Promega Cell Titer Glo assay system. The bar indicates 50% cell growth inhibition. (See FIG. 10).

### Example 11

### Various Concentrations of Flori on Cells Over Stimulated With IL-2

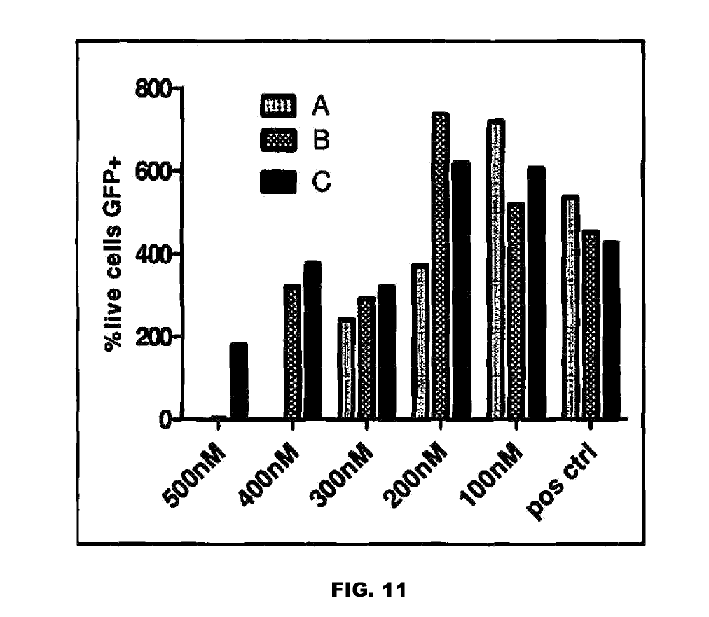

Human peripheral blood mononuclear cells (PBMCs) purified from a single donor were prepared in triplicate (A,B,C), cultured in enriched media (RPM1+20% FBS) and stimulated pre-infection (24 hrs) and every 3 days for three weeks with 2 ng/ml of phytoheaemagglutinin (PHA),1 U/ml IL-2 and 2% FBS by volume. 24 hours following the initial stimulation the samples were incubated for one hour with the indicated amounts of NSC166641 and then infected with 300 ng HIV-1 (wt). Samples were analyzed via ELISA p24. Activity shows wide variability in HIV p24 expression from the same donor. (See FIG. 11).

### Example 12

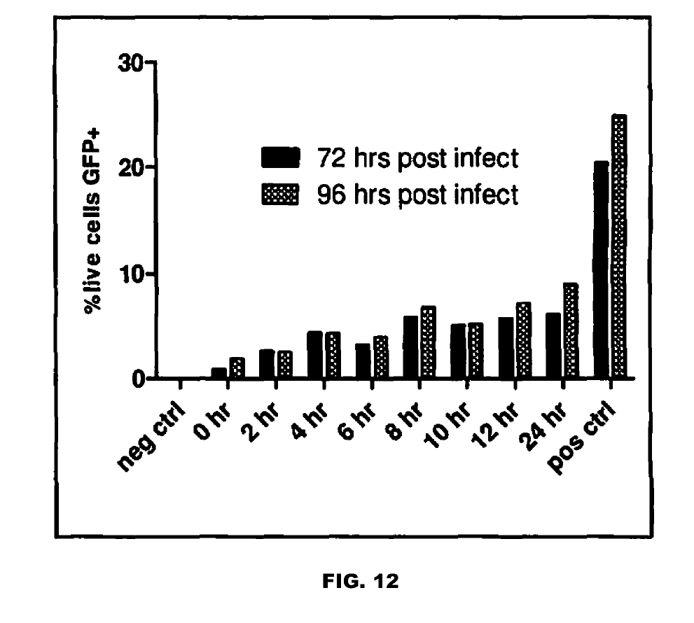

Reagent activity post infection with HIV-1 virus (wt). G11 (GFP) HIV reporter cells (200K) were seeded in 5 ml falcon tubes (RPMI+10% FBS). 1 uM of NSC166641 was added post infection at the hours indicated with HIV-1 (wt)(250 ng). Samples were stained with propidium iodide and analyzed for GFP expression via flow cytometer 72 and 96 hours post infection. (See FIG. 12).

### Example 13

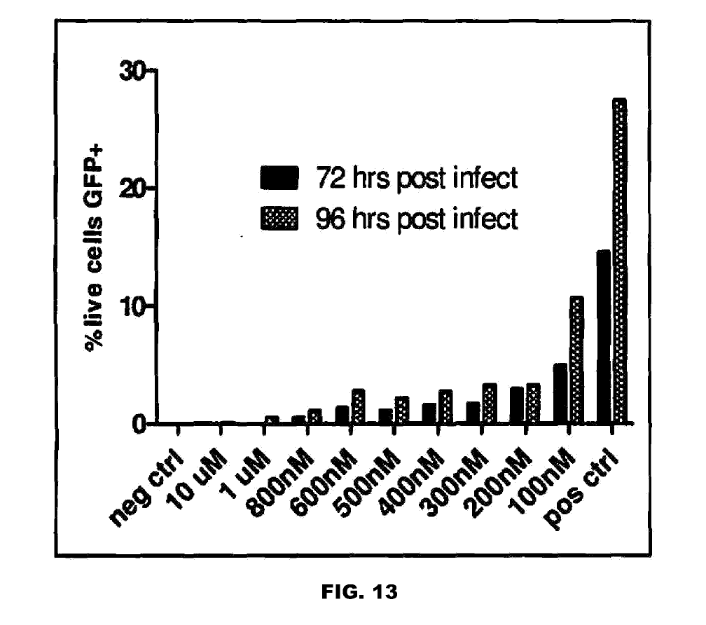

G11 HIV reporter cells (200K) were seeded in 5 ml falcon tubes (RPMI+10% FBS) and incubated 1 hour with the indicated amounts of NSC 166641 before infection with HIV-1 (wt) virus (250 ng) Samples were stained with propidium iodide and analyzed for GFP expression via flow cytometer 72 and 96 hours post infection. (See FIG. 13).

### Example 14

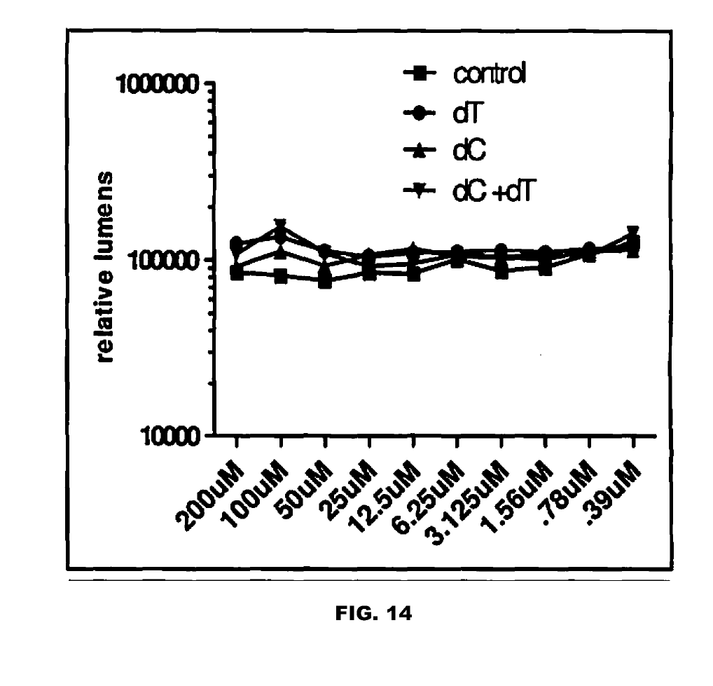

Clone 64 luciferase reporter CEM-ss cells (50K) were seeded (RPMI+10% FBS) in 96 well plates incubated for one hour with deoxycytidine (dC), deoxythymidine (dT) and both dC and dT before infection with 20 ul (20 ng) of vsv-g pseudotyped HIV-1 virus. Results were analyzed via luminometer at 48 hours post infection. (See FIG. 14).

### Example 15

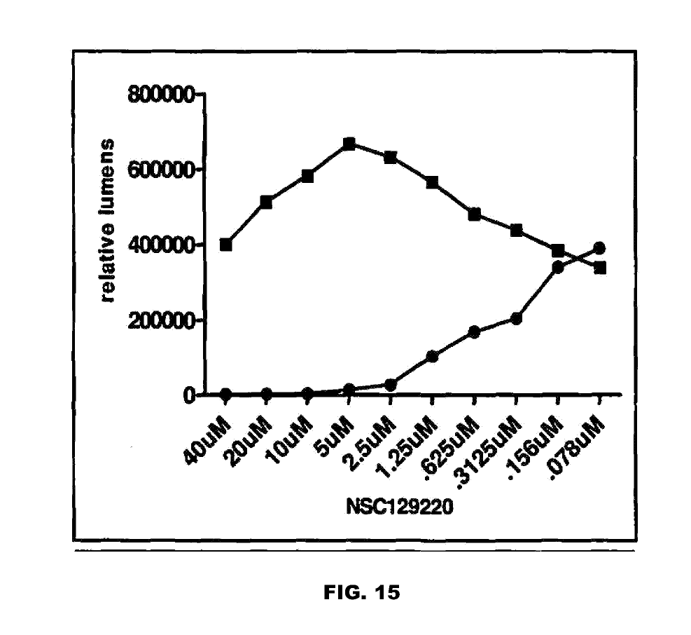

Clone 64 luciferase reporter CEM-ss cells (50K) were seeded (RPMI+10% FBS) in 96 well plates incubated for one hour with the indicated amounts of NSC129220 and with (box) and without (dot) 200 uM deoxycytidine (dC) before infection with 20 ul (20 ng) of vsv-g pseudotyped HIV-1 virus. Results were analyzed via luminometer 48 hours post infection. Addition of dC is able to rescue luciferase expression in treated cells. (50K) were seeded (RPMI+10% FBS) in 96 well plates incubated for one hour with the indicated amounts of NSC166641 and with (box)/without (dot) 200 uM deoxycytidine (dC) before infection with 20 ul (20 ng) of vsv-g pseudotyped HIV-1 virus. Results were analyzed via luminometer 48 hours post infection. Addition of dC appears to be able to rescue luciferase expression in treated cells. (See FIG. 15).

### Example 16

### Dose Response

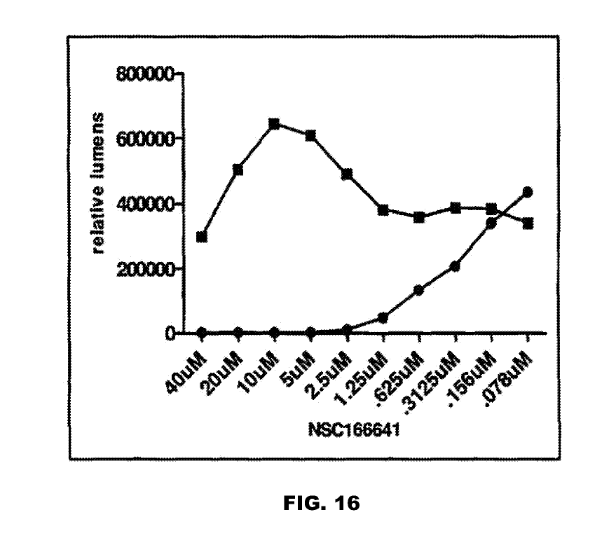

Clone 64 luciferase reporter CEM-ss cells (50K) were seeded (RPMI+10% FBS) in 96 well plates incubated for one hour with the indicated amounts of NSC166641 and (box) and without (dot) 200 uM deoxycytidine (dC) before infection with 20 ul (20 ng) of vsv-g pseudotyped HIV-1 virus. Results were analyzed via luminometer 48 hours post infection. Addition of dC is able to rescue luciferase expression in treated cells. (See FIG. 16).

### Example 17

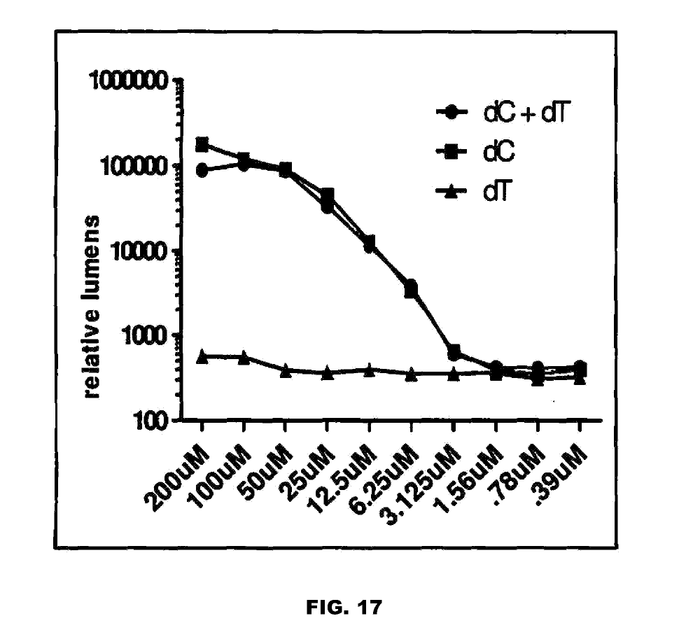

Clone 64 luciferase reporter CEM-ss cells (50K) were seeded (RPMI+10% FBS) in 96 well plates treated with 10 uM NSC166641 and the indicated amounts of dC, dT or both dC and dT before infection with 20 ul (20 ng) of vsv-g pseudotyped HIV-1 virus. Results were analyzed via luminometer 48 hours post infection. The results indicate that the addition of dC but not dT may be able to rescue luciferase expression in treated cells. (See FIG. 17).

### Example 18

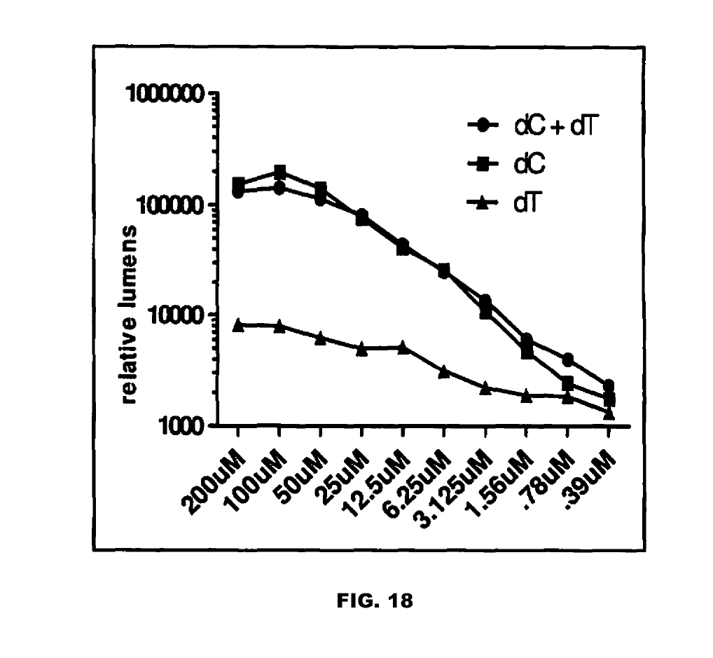

Clone 64 luciferase reporter CEM-ss cells (50K) were seeded (RPMI+10% FBS) in 96 well plates treated with 1 μM NSC166641 and the indicated amounts of dC, dT or both dC and dT before infection with 20 ul (20 ng) of vsv-g pseudotyped HIV-1 virus. Results were analyzed via luminometer 48 hours post infection. The results indicate that addition of dC but not dT may be able to rescue luciferase expression in treated cells. (See FIG. 18).

### Example 19

### Characterizing Flori

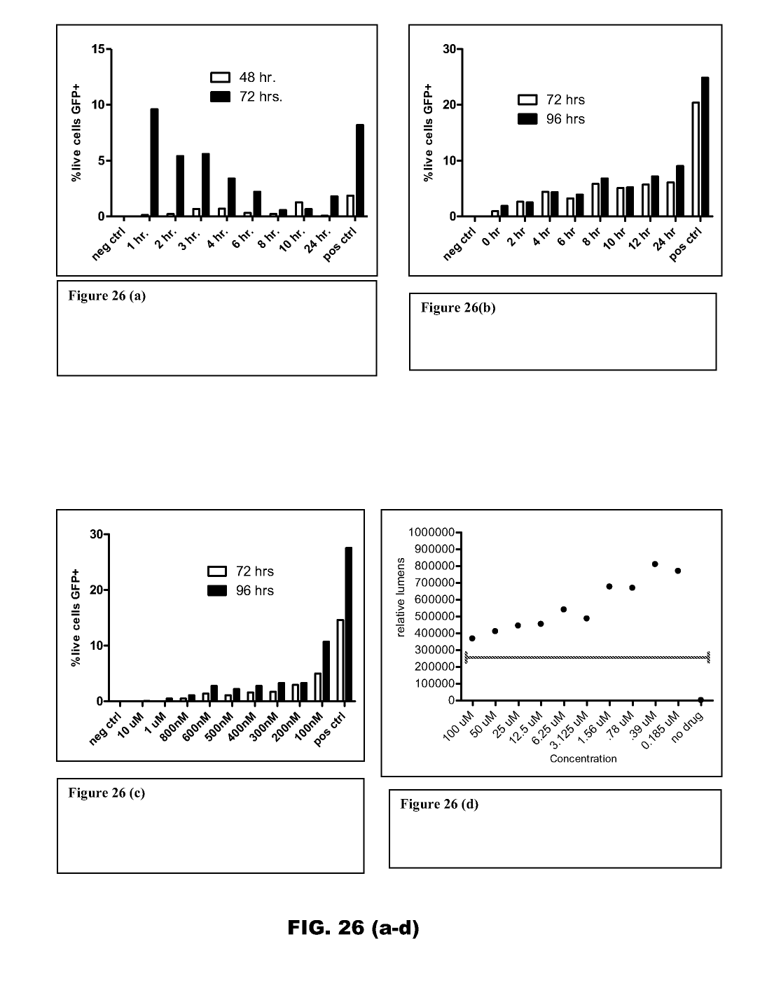

The percentage of live G11 cells expressing GFP following 1 uM Flori pre-incubation and washout before infection with HIV (wt) at the hours indicated. (See FIG. 26 (a).

The percentage of live G11 cells expressing GFP following infection with HIV (wt) and then treated AFTER infection with 1 uM Flori at the hours indicated. (See FIG. 26(b).

The dose-dependent effect of Flori was analyzed. G11 cells were incubated 24 hours before infection with indicated amounts of Flori before infection with HIV (wt) at the hours indicated. The percentage of live G11 cells expressing GFP was measured. (See FIG. 26 (c).

The IC50 of Flori was measured. A 48 hour Flori IC50 in PBMC assay as measured by the Promega CellTiter-Glo® IC50 (50% inhibition concentration) was completed. The red bar denotes 50% inhibition. (See FIG. 26(d).

### Example 20

### Effectiveness of Cyclocytidines

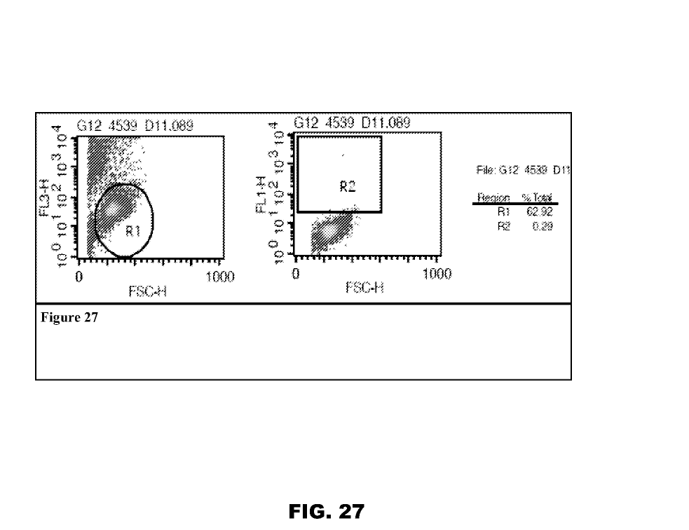

Example FACS dot plots of live G11 cells expressing HIV induced expression of GFP. Well 4539 D-11 (NSC129220) confirmation of initial luciferase screen and inhibition of HIV. (See FIG. 27).

### Example 21

### The Dose-Dependent Effect of Flori

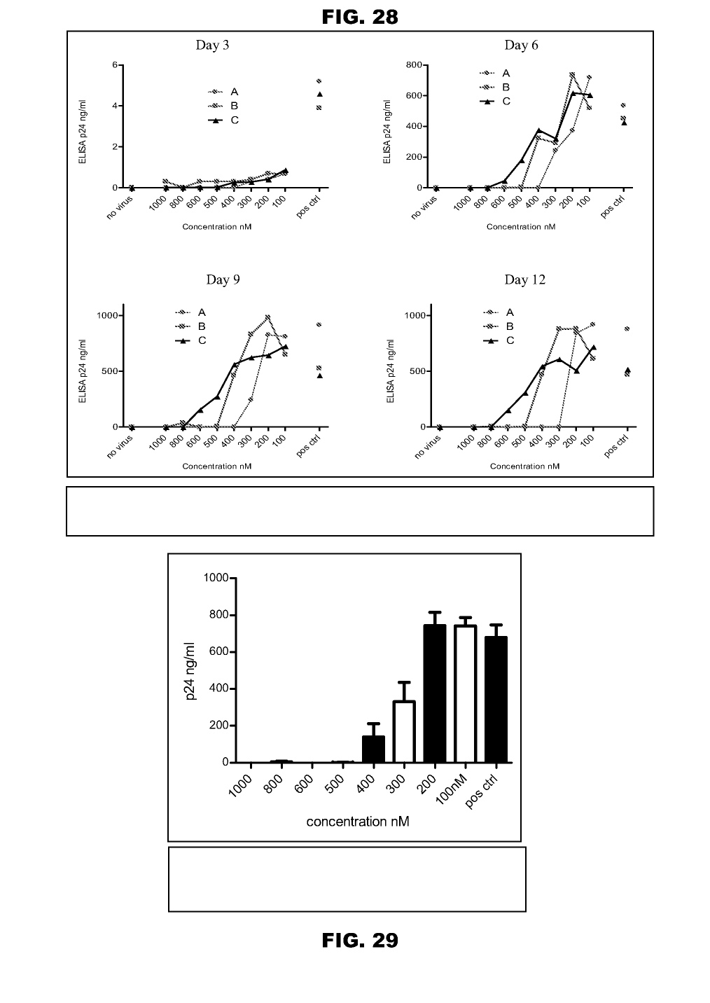

The dose-dependent effect of Flori was measured. The dose-dependent effect of Flori concentrations on p24 levels in HIV (wt) infected PBMCs samples A, B and C from the same donor stimulated every two days with IL-2 and PHA and grown in enriched medium (+20% FBS) was measured at days 3, 6, 9 and 12. (See FIG. 28).

### Example 22

### The Dose-Dependent Effect of Flori

Final analysis of Flori dose dependent effect on p24 levels in PBMC samples A, B, and C from the same donor at days 6, 9 and 12. (See FIG. 29).

### Example 23

### Screening the DTP Diversity Set For Anti-Retroviral Activity

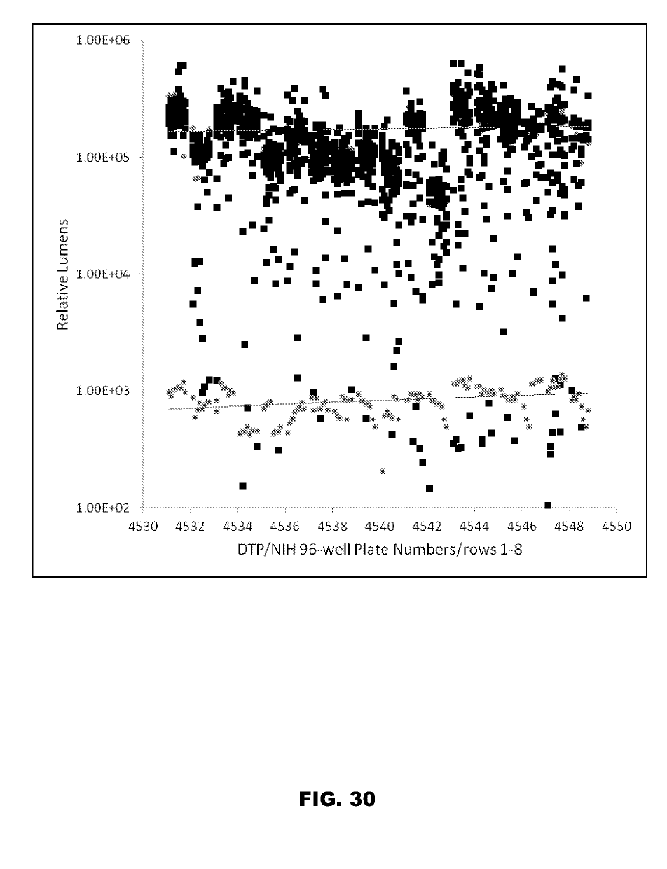

2 uM/0.4% DMSO DTP diversity set screen II. HIV inhibition was indicated by luminescence quench (<1×10⁴). Clone 64 cells were seeded (5×10⁵) per well, incubated with test reagents for 60 minutes before infection with 20 ul of HIV-1 (1,200 ng/ml) vsv-g pseudotyped virus, and incubated for 48 hours 37° C. Cells were lysed with 10× buffer and lumens were measured after injection with 30 ul of 10× luciferase buffer. Note that plates 4546 and 4548 were only seeded with 40 reagents. Trend lines represent negative (lower)/positive (upper) controls respectively. (See FIG. 30).

### Example 24

### Head-To-Head Comparison of AZT with Flori

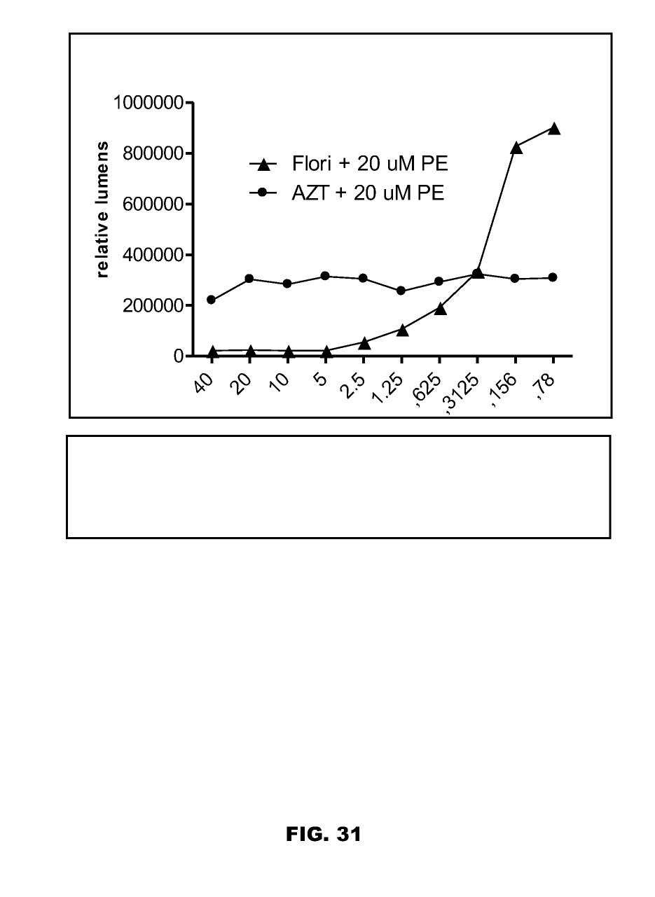

Clone 64 cells were treated with 20 μM phorbol ester (PE) (NSC339875). A dose dependent effect of Flori and AZT in (μM) on Clone 64 cells was observed. (See FIG. 31).

### Example 25

### Head-To-Head Comparisons of Flori, Dideoxycytidine, Dideoxyfluorocytidine, Thiacytidine (3TC), Cyclopentosine, and Mycophenolic acid in a Rev Dependent Luciferase Reporter Cell Line

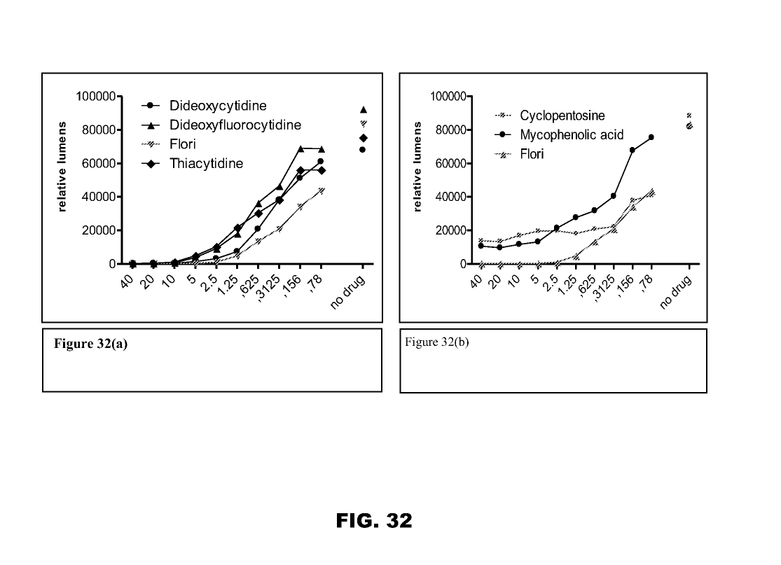

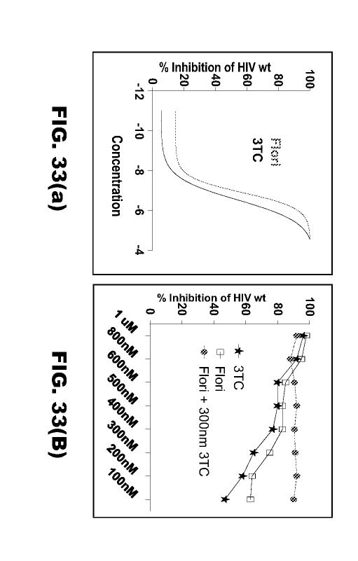

Several approved anti-HIV compounds were compared in a head-to-head test. The dose-dependent comparison was carried out in a Clone 64 HIV induced luciferase assay (concentration u M). Flori was first compared to Dideoxycytidine, Dideoxyfluorocytidine and Thiacytidine, Cyclopentosine, and Mycophenolic acid (See FIG. 32(a). Flori was then compared to Cyclopentosine, and Mycophenolic acid in Clone 64 HIV induced luciferase assay (concentration uM). (See FIG. 32(b).

### Example 26

### Measuring the Relative Potency and Cooperation of Flori and the Lamivudine (3TC)

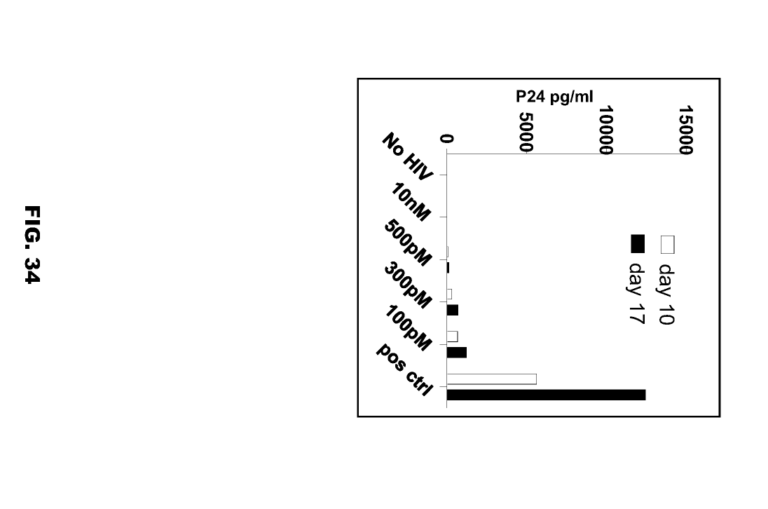

The dose response curve of Flori vs. 3TC was measured. Luciferase reporter cells incubated with the indicated amounts of reagent 12 hrs. before infection with HIV lwt. Luciferase expression measured via luminometer 48 hrs. post infection. Percent inhibition as compared to positive control. (See FIG. 33(a).

A combination assay of Flori and 3TC was performed. Flori and 3Tc were combined by mixing. The combination was contacted with cells by administering the compound to the cells. GFP reporter cells were incubated with the indicated amounts of reagent 12 hours before infection with HIV lwt. GFP expression was measured in viable cell populations via flow cytometer at 96 hours post-infection. Percent inhibition as compared to positive control was measured. (See FIG. 33(b).

### Example 27

### Latent Virus Activation and Flori

Flori inhibition of HIV infection of resting CD4T cells was measured. Human resting CD4T cells were treated at one hour with the indicated amounts of Flori and then infected with HIV 1 (wt). At day five, resting cells were activated with CD3/CD28 beads (4×10⁸). Results were analyzed via p24 ELISA at days 10 and 17. (See FIG. 34).

### Example 28

### Inhibition of dCK With Flori

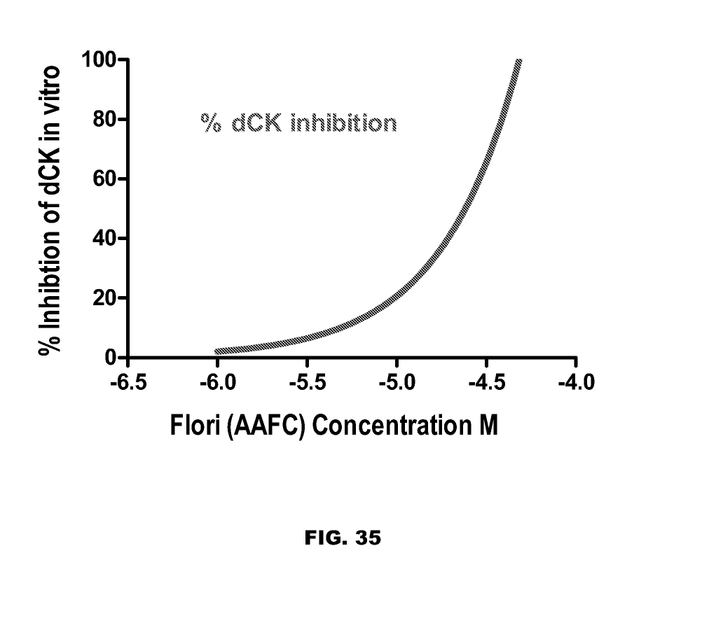

Inhibiting the activity of the dCK enzyme is useful. In order to demonstrate the effectiveness of Flori, a Flori (AAFC)—dCK inhibition curve was generated through experimental testing. Human recombinant dCK activity was characterized using deoxyinosine (dIR) as a substrate. Assays were performed in duplicate in 200 μL of reaction mixture (96-well plate). Reactions were started by adding dIR at various concentrations and the percent inhibition was determined as compared to the positive control via spectrophotometer. The indicated dCK IC50 of AAFC was measured to be 3.2×10⁻⁵M. (See FIG. 35).

### Example 29

### Methods of Making Compositions Containing 5-Fluoro-2,2′-Cyclocytidine, O2,2′-Cyclocytidine, O2,2′-Cyclocytidine Monoacetate, and/or O2,2′-Cyclocytidine Hydrochloride Useful in the Treatment of HIV Infection

Antiretroviral drugs may be combined into a cocktail which is useful in the treatment of HIV. One or more of 5-Fluoro-2,2′-cyclocytidine, O2,2′-cyclocytidine, O2,2′-cyclocytidine monoacetate, and/or O2,2′-cyclocytidine hydrochloride are combined with other antiretroviral drugs. Antiretroviral drugs may include, among other compounds, those set forth herein. By way of non-limiting example, one or more of 5-Fluoro-2,2′-cyclocytidine, O2,2′-cyclocytidine, O2,2′-cyclocytidine monoacetate, and O2,2′-cyclocytidine hydrochloride may be combined with one or more of nucleoside and nucleotide reverse transcriptase inhibitors (NRTI), non-nucleoside reverse transcriptase inhibitors, protease inhibitors, integrase inhibitors, entry inhibitors, and maturation inhibitors.

By way of non-limiting example, one or more of 5-Fluoro-2,2′-cyclocytidine, O2,2′-cyclocytidine, O2,2′-cyclocytidine monoacetate, and/or O2,2′-cyclocytidine hydrochloride may be combined with one or more of emtricitabine (an NRTI), tenofovir (an NRTI), efavirenz (a NNRTI), raltegravir (an integrase inhibitor), darunavir (a protease inhibitor), ritonavir (a protease inhibitor), atazanavir (a protease inhibitor), zidovudine, lamivudine, abacavir, lopinavir, stavudine, lamivudine, and nevirapine, combivir (Glaxo Smith Kline), emtriva (Gilead sciences), epivir (GlaxoSmithKline), Epivir (Glaxo Smith Klein), Epzicom (Glaxo Smith Kline), Hivid (Hoffman-La Roche), Retrovir, Trizivir (Glaxo Smith Kline), lamivudine and zidovudine (GlaxoSmithKline), Emtricitabine (Gilead Sciences), lamivudine, 3TC (GlaxoSmithKline), abacavir/lamivudine (GlaxoSmithKline), zalcitabine, ddC, dideoxycytidine (Hoffmann-La Roche), zidovudine, AZT, azidothymidine, ZDV (Glaxo Smith Kline), abacavir, zidovudine, and lamivudine (Glaxo Smith Kline), tenofovir disoproxil/emtricitabine (Gilead Sciences, Inc.), enteric coated didanosine (Bristol Myers-Squibb), didanosine, ddI, dideoxyinosine (Bristol Myers-Squibb), tenofovir disoproxil fumarate (Gilead Sciences), stavudine, d4T (Bristol Myers-Squibb), and abacavir (Glaxo Smith Kline).

### Example 30

### Combinations of Flori (NSC 166641) and One Or More Antiretroviral Drugs and Including Lamivudine (3TC) For The Treatment of HIV Infection

Combinations of Flori (NSC 166641)) with one or more antiretroviral drugs are useful in the treatment of HIV infection. (See FIG. 33). The FDA approved antiretroviral drug Lamivudine (3TC) was combined (See FIG. 33(b), see the top trend line) with Flori (NSC 166641) in varying dosages. Combination studies of Flori (NSC 166641) and Lamivudine revealed that the two drugs may complement each other.

Additionally, experimental results indicate that Flori (NSC 166641) may not counteract the efficacy of Lamivudine (3TC) and vice versa. Furthermore, the two drugs were more efficacious at lower concentrations in combinations than the two drugs demonstrated independently (FIG. 33(b)). Both cytidine analogs and 3TC may be phosphorylated by dCK. These results indicate that Flori (NSC 166641) may improve the efficacy of other approved antiretroviral drugs including, those drugs disclosed herein.

In this specification, “a” and “an” and similar phrases are to be interpreted as “at least one” and “one or more.” References to “an” embodiment in this disclosure are not necessarily to the same embodiment.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, it will be apparent that cyclocytidines may be substituted for HIV cocktail components that are more expensive or less potent. Thus, the present embodiments should not be limited by any of the above-described exemplary embodiments.

In addition, it should be understood that any figures which highlight the functionality and advantages, are presented for example purposes only. The disclosed architecture is sufficiently flexible and configurable, such that it may be utilized in ways other than that shown. For example, the process for treating HIV may include the use of therapeutic composition breakdown products having medicinal benefits distinct from the compound administered in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6. Claims that do not expressly include the phrase “means for” or “step for” are not to be interpreted under 35 U.S.C. 112, paragraph 6.

What is claimed is:

1. A composition comprising one or more antiretroviral drugs and one or more of 5-fluoro-2,2′-cyclocytidine, O2,2′-Cyclocytidine, the compounds set forth in FIG. 19, and pharmaceutically acceptable prodrugs or salts salt thereof respectively.

2. The composition according to claim 1, wherein said one or more antiretroviral drugs comprises one or more of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor.

3. The composition according to claim 1, wherein said one or more antiretroviral drugs comprises one or more of lamivudine, tenofovir, raltegravir, darunavir, ritonavir, atazanavir, zidovudine, abacavir, lopinavir, stavudine, and nevirapine.

4. The composition according to claim 1, further comprising about 2 to about 99 antiretroviral drugs.

5. The composition of claim 2, wherein said combining is by one or more of mixing, oral administration, parenteral administration, transdermal administration, buccal administration, nasal administration, mucosal administration, and sublingual administration.

6. A process to treat an infection of human immunodeficiency virus (HIV) comprising: administering to a patient a therapeutically effective amount of a first compound wherein said first compound is at least one of O2,2′-Cyclocytidine (CycloC), the compounds set forth in FIG. 19, and pharmaceutically acceptable prodrugs or salts salt thereof respectively.

7. The process according to claim 6, further comprising administering to the patient a therapeutically effective amount of at least one second compound wherein said at least one second compound is selected from the group consisting of 5-fluoro-2,2′-cyclocytidine, a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, a maturation inhibitor, tenofovir, raltegravir, darunavir, ritonavir, atazanavir, zidovudine, lamivudine, abacavir, lopinavir, stavudine, lamivudine, and nevirapine.

8. The process according to claim 6 or 7, wherein a cell is contacted with at least one of O2,2′-Cyclocytidine, the compounds set forth in FIG. 19, and pharmaceutically acceptable prodrugs or salts salt thereof respectively.

9. The process according to claim 6 or 7, wherein the at least one of the at least one of O2,2′-Cyclocytidine, the compounds set forth in FIG. 19, and pharmaceutically acceptable prodrugs or salts salt thereof respectively thereof is administered at least one of orally, parenterally, transdermally, bucally, nasally, mucosally, and sublingually.

10. The process according to claim 9, wherein the therapeutically effective amount is from about 1 ng to about 1000 mg per day.

11. A process to treat an infection of human immunodeficiency virus comprising administering to the patient a therapeutically effective amount of at least one of 5-fluoro-2,2′-cyclocytidine, the compounds set forth in FIG. 19, and pharmaceutically acceptable prodrugs or salts salt thereof respectively.

12. The process according to claim 11, further comprising administering to the patient a therapeutically effective amount of at least one second compound wherein said at least one second compound is selected from the group consisting of lamivudine, 5-fluoro-2,2′-cyclocytidine, a nucleoside reverse transcriptase inhibitor, a nucleotide reverse transcriptase inhibitor, a non-nucleoside reverse transcriptase inhibitors, a protease inhibitor, an integrase inhibitor, an entry inhibitor, a maturation inhibitor, tenofovir, raltegravir, darunavir, ritonavir, atazanavir, zidovudine, abacavir, lopinavir, stavudine, and nevirapine.

13. The process according to claim 11 or 12, wherein at least one of the at least one of 5-fluoro-2,2′-cyclocytidine, pharmaceutically acceptable prodrug thereof and salt thereof is administered at least one of orally, parenterally, transdermally, bucally, nasally, mucosally, and sublingually.

14. The process according to claim 11, wherein the therapeutically effective amount is from about 1 ng to about 1000 mg per day.

15. A method of making a composition comprising:
combining one or more antiretroviral drugs with
at least one of O2,2′-Cyclocytidine, the compounds set forth in FIG. 19, 5-fluoro-2, 2′-cyclocytidine, and pharmaceutically acceptable prodrugs or salts salt thereof respectively.

16. The method according to claim 15, wherein said one or more antiretroviral drugs comprises one or more of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor.

17. The method according to claim 15, wherein said one or more antiretroviral drugs comprises one or more of lamivudine, tenofovir, raltegravir, darunavir, ritonavir, atazanavir, zidovudine, abacavir, lopinavir, stavudine, and nevirapine.

18. The method according to claim 15, wherein said combining is by one or more of mixing, ingestion, oral administration, parenteral administration, transdermal administration, buccal administration, nasal administration, mucosal administration, and sublingual administration.
